SEQUENCE LISTING (96 sequences)

SEQ ID NO: 1 - protein, 7 aa, Human immunodeficiency virus
KRRQRRR

SEQ ID NO: 2 - protein, 16 aa, Unknown Organism
  Description of Unknown Organism Unknown Penetratin PTD peptide
RQIKIWFQNRRMKWKK

SEQ ID NO: 3 - protein, 34 aa, herpes simplex virus
DAATATRGRSAASRPTERPRAPARSASRPRRPVD

SEQ ID NO: 4 - protein, 16 aa, Homo sapiens
AAVALLPAVLLALLAP

SEQ ID NO: 5 - protein, 16 aa, Homo sapiens
AAVLLPVLLPVLLAAP

SEQ ID NO: 6 - protein, 15 aa, Homo sapiens
VTVLALGALAGVGVG

SEQ ID NO: 7 - protein, 17 aa, Artificial Sequence
  Description of Artificial Sequence gp41 fusion peptide
GALFLGWLGAAGSTMGA

SEQ ID NO: 8 - protein, 17 aa, Caiman crocodilus
MGLGLHLLVLAAALQGA

SEQ ID NO: 9 - protein, 24 aa, Unknown Organism
  Description of Unknown Organism hCT-derived peptide
LGTYTQDFNKFHTFPQTAIGVGAP

SEQ ID NO: 10 - protein, 26 aa, Unknown Organism
  Description of Unknown Organism Unknown transportan peptide
GWTLNSAGYLLKINLKALAALAKKIL

SEQ ID NO: 11 - protein, 16 aa, Unknown Organism
  Description of Unknown Organism Unknown loligomer peptide
TPPKKKRKVEDPKKKK

SEQ ID NO: 12 - protein, 7 aa, Unknown Organism
  Description of Unknown Organism Unknown Arginine peptide
RRRRRRR

SEQ ID NO: 13 - protein, 18 aa, Artificial Sequence
  Description of Artificial Sequence Amphiphilic model peptide
KLALKLALKALKAALKLA

SEQ ID NO: 14 - protein, 16 aa, Influenza virus
GLFGAIAGFIENGWEG

SEQ ID NO: 15 - protein, 16 aa, Sendai virus
FFGAVIGTIALGVATA

SEQ ID NO: 16 - protein, 16 aa, respiratory syncytial virus
FLGFLLGVGSAIASGV

SEQ ID NO: 17 - protein, 16 aa, Human immunodeficiency virus
GVFVLGFLGFLATAGS

SEQ ID NO: 18 - protein, 16 aa, Ebola virus
GAAIGLAWIPYFGPAA

SEQ ID NO: 19 - protein, 56 aa, Unknown Organism
  Description of Unknown Organism Exemplary zinc finger motif
ACTCPYCKDSEGRGSGDPGKKKQHICHIQGCGKVYGKTSHLRAHLRWHTGERPFMC

SEQ ID NO: 20 - protein, 54 aa, Unknown Organism
  Description of Unknown Organism Exemplary zinc finger motif
ACTCPNCKDGEKRSGEQGKKKHVCHIPDCGKTFRKTSLLRAHVRLHTGERPFVC

SEQ ID NO: 21 - protein, 55 aa, Unknown Organism
  Description of Unknown Organism Exemplary zinc finger motif
ACTCPNCKEGGGRGTNLGKKKQHICHIPGCGKVYGKTSHLRAHLRWHSGERPFVC

SEQ ID NO: 22 - protein, 56 aa, Unknown Organism
  Description of Unknown Organism Exemplary zinc finger motif
ACSCPNCREGEGRGSNEPGKKKQHICHIEGCGKVYGKTSHLRAHLRWHTGERPFIC

SEQ ID NO: 23 - protein, 60 aa, Unknown Organism
  Description of Unknown Organism Exemplary zinc finger motif
RCTCPNCTNEMSGLPPIVGPDERGRKQHICHIPGCERLYGKASHLKTHLRWHTGERPFLC

SEQ ID NO: 24 - protein, 58 aa, Unknown Organism
  Description of Unknown Organism Exemplary zinc finger motif
TCDCPNCQEAERLGPAGVHIRKKNIHSCHIPGCGKVYGKTSHLKAHLRWHTGERPFVC

SEQ ID NO: 25 - protein, 53 aa, Unknown Organism
  Description of Unknown Organism Exemplary zinc finger motif
RCTCPNCKAIKHGDRGSQHTHLCSVPGCGKTYKKTSHLRAHLRKHTGDRPFVC

SEQ ID NO: 26 - protein, 56 aa, Unknown Organism
  Description of Unknown Organism Exemplary zinc finger motif
PQISLKKKIFFFIFSNFRGDGKSRIHICHLCNKTYGKTSHLRAHLRGHAGNKPFAC

SEQ ID NO: 27 - protein, 23 aa, Artificial Sequence
  Description of Artificial Sequence Synthetic formula peptide
CXXXXCXXXXXXXXXXXXHXXXH

SEQ ID NO: 28 - protein, 28 aa, Unknown Organism
  Description of Unknown Organism Exemplary Peptide
KETWWETWWTEWSQPGRKKRRQRRRPPQ

SEQ ID NO: 29 - protein, 28 aa, Unknown Organism
  Description of Unknown Organism Exemplary Peptide
WWTWWWWWWWEWSQPKKKKRRRRRRPPQ

SEQ ID NO: 30 - protein, 23 aa, Unknown Organism
  Description of Unknown Organism Exemplary Peptide
WWWWWWWWWWSQPKKKKKKKKKK

SEQ ID NO: 31 - protein, 23 aa, Unknown Organism
  Description of Unknown Organism Exemplary Peptide
WWWWWWWWWWSQPRRRRRRRRRR

SEQ ID NO: 32 - protein, 28 aa, Unknown Organism
  Description of Unknown Organism Exemplary Peptide
KWWWWWWWWWEWSQPKKKKRRRRRRKKK

SEQ ID NO: 33 - protein, 20 aa, Unknown Organism
  Description of Unknown Organism Exemplary Peptide
KHKHKHKHKHKHKHKHKHKH

SEQ ID NO: 34 - protein, 20 aa, Unknown Organism
  Description of Unknown Organism Exemplary Peptide
KHKHKHKHKHKHKHKHKHKH

SEQ ID NO: 35 - protein, 30 aa, Unknown Organism
  Description of Unknown Organism Exemplary Peptide
HHHHHHRSVCRQIKICRRRGGCYKCTXRPY

SEQ ID NO: 36 - protein, 29 aa, Unknown Organism
  Description of Unknown Organism Exemplary Peptide
KKKKKKKKKGLFGAIAGFIEXGWEGMIDG

SEQ ID NO: 37 - protein, 36 aa, Unknown Organism
  Description of Unknown Organism Exemplary Peptide
KGSKKAVTKAQKKEGKKRKRSRKESYSVYVYKVLKQ

SEQ ID NO: 38 - protein, 9 aa, Unknown Organism
  Description of Unknown Organism Exemplary Peptide
RRRRRRRRR

SEQ ID NO: 39 - protein, 20 aa, Unknown Organism
  Description of Unknown Organism Exemplary Peptide
KKKKKKKKKKKKKKKKKKKK

SEQ ID NO: 40 - protein, 18 aa, Unknown Organism
  Description of Unknown Organism Exemplary Peptide
RRRRRRRRRRRRRRRRRR

SEQ ID NO: 41 - protein, 30 aa, Unknown Organism
  Description of Unknown Organism Exemplary Peptide
KKKKKKKKKKKKKKKKKKKKKKKKKKKKKK

SEQ ID NO: 42 - protein, 36 aa, Unknown Organism
  Description of Unknown Organism Exemplary Peptide
KGSKKAVTKAQKKDGKKRKRSRKESYSVYVYKVLKQ

SEQ ID NO: 43 - protein, 18 aa, Unknown Organism
  Description of Unknown Organism Exemplary Peptide
KLALKLALKALKAALKLA

SEQ ID NO: 44 - DNA, 21 bp, Artificial Sequence
  Description of Combined DNA/RNA Molecule Synthetic siRNA
ccgucagccgauuugcuautt

SEQ ID NO: 45 - DNA, 21 bp, Artificial Sequence
  Description of Combined DNA/RNA Molecule Synthetic siRNA
auagcaaaucggcugacggtt

SEQ ID NO: 46 - DNA, 21 bp, Artificial Sequence
  Description of Combined DNA/RNA Molecule Synthetic siRNA
gggucggaacccaagcuuatt

SEQ ID NO: 47 - DNA, 21 bp, Artificial Sequence
  Description of Combined DNA/RNA Molecule Synthetic siRNA
uaagcuuggguuccgacccta

SEQ ID NO: 48 - DNA, 21 bp, Artificial Sequence
  Description of Combined DNA/RNA Molecule Synthetic siRNA
cgggacucuagcauacuuatt

SEQ ID NO: 49 - DNA, 21 bp, Artificial Sequence
  Description of Combined DNA/RNA Molecule Synthetic siRNA
uaaguaugcuagagucccgtt

SEQ ID NO: 50 - DNA, 21 bp, Artificial Sequence
  Description of Combined DNA/RNA Molecule Synthetic siRNA
acugacagccagacagcgatt

SEQ ID NO: 51 - DNA, 21 bp, Artificial Sequence
  Description of Combined DNA/RNA Molecule Synthetic siRNA
ucgcugucuggcugucagutt

SEQ ID NO: 52 - DNA, 21 bp, Artificial Sequence
  Description of Combined DNA/RNA Molecule Synthetic siRNA
agacagcgaccaaaagaautt

SEQ ID NO: 53 - DNA, 21 bp, Artificial Sequence
  Description of Combined DNA/RNA Molecule Synthetic siRNA
auucuuuuggucgcugucutt

SEQ ID NO: 54 - DNA, 21 bp, Artificial Sequence
  Description of Combined DNA/RNA Molecule Synthetic siRNA
augaagaucuguuccaccatt

SEQ ID NO: 55 - DNA, 21 bp, Artificial Sequence
  Description of Combined DNA/RNA Molecule Synthetic siRNA
ugguggaacagaucuucautt

SEQ ID NO: 56 - DNA, 21 bp, Artificial Sequence
  Description of Combined DNA/RNA Molecule Synthetic siRNA
gaucuguuccaccauugaatt

SEQ ID NO: 57 - DNA, 21 bp, Artificial Sequence
  Description of Combined DNA/RNA Molecule Synthetic siRNA
uucaaugguggaacagauctt

SEQ ID NO: 58 - DNA, 21 bp, Artificial Sequence
  Description of Combined DNA/RNA Molecule Synthetic siRNA
gcaauugaggagugccugatt

SEQ ID NO: 59 - DNA, 21 bp, Artificial Sequence
  Description of Combined DNA/RNA Molecule Synthetic siRNA
ucaggcacuccucaauugctt

SEQ ID NO: 60 - DNA, 21 bp, Artificial Sequence
  Description of Combined DNA/RNA Molecule Synthetic siRNA
uugaggagugccugauuaatt

SEQ ID NO: 61 - DNA, 21 bp, Artificial Sequence
  Description of Combined DNA/RNA Molecule Synthetic siRNA
uuaaucaggcacuccucaatt

SEQ ID NO: 62 - DNA, 21 bp, Artificial Sequence
  Description of Combined DNA/RNA Molecule Synthetic siRNA
ggaucuuauuucuucggagtt

SEQ ID NO: 63 - DNA, 21 bp, Artificial Sequence
  Description of Combined DNA/RNA Molecule Synthetic siRNA
cuccgaagaaauaagaucctt

SEQ ID NO: 64 - DNA, 21 bp, Artificial Sequence
  Description of Combined DNA/RNA Molecule Synthetic siRNA
cuacacaaaucagcgauuutt

SEQ ID NO: 65 - DNA, 21 bp, Artificial Sequence
  Description of Combined DNA/RNA Molecule Synthetic siRNA
aaaucgcugauuuguguagtc

SEQ ID NO: 66 - protein, 125 aa, Artificial Sequence
  Description of Artificial Sequence Synthetic Polypeptide
MPEPAKSAPAPKKGSKKAVTKAQKKDSKKRKRSRKESYSVYVYKVLKVHPDTGISSKAMGIMNSFVNDIFERIAGEASRLAHYNKRSTITSREIQTAVRLLLPGELAKHAVSEGTKAVTKYTSSK

SEQ ID NO: 67 - protein, 36 aa, Artificial Sequence
  Description of Artificial Sequence Synthetic Peptide
KGSKKAVTKAQKKDGKKRKRSRKESYWVYVYKVLKQ

SEQ ID NO: 68 - protein, 36 aa, Artificial Sequence
  Description of Artificial Sequence Synthetic Peptide
KGSKKAVTKAQKKDGKKRKRSRKWSYSVYVYKVLKQ

SEQ ID NO: 69 - protein, 36 aa, Artificial Sequence
  Description of Artificial Sequence Synthetic Peptide
KGSKKAVTKAQKKDGKKRKRSRKFSYSVYVYKVLKQ

SEQ ID NO: 70 - protein, 36 aa, Artificial Sequence
  Description of Artificial Sequence Synthetic Peptide
KGSFKAVTKAQKKDGKKRKRSFKFSYSVYVYKVLKQ

SEQ ID NO: 71 - protein, 35 aa, Artificial Sequence
  Description of Artificial Sequence Synthetic Peptide
KGSFKAVTKAQKKFGKKRKRSRKSFSVYVYKVLKQ

SEQ ID NO: 72 - protein, 23 aa, Artificial Sequence
  Description of Artificial Sequence Synthetic Peptide
KGSKKAVTKAQKKDGKKRKRSRK

SEQ ID NO: 73 - protein, 25 aa, Artificial Sequence
  Description of Artificial Sequence Synthetic Peptide
KKDGKKRKRSRKESYSVYVYKVLKQ

SEQ ID NO: 74 - protein, 15 aa, Artificial Sequence
  Description of Artificial Sequence Synthetic Peptide
RKESYSVYVYKVLKQ

SEQ ID NO: 75 - protein, 36 aa, Artificial Sequence; identical to SEQ ID NO: 42
  Description of Artificial Sequence Synthetic Peptide

SEQ ID NO: 76 - protein, 33 aa, Artificial Sequence
  Description of Artificial Sequence Synthetic Peptide
KKAVTKAQKKDGKKRKRSRKESYSVYVYKVLKQ

SEQ ID NO: 77 - protein, 30 aa, Artificial Sequence
  Description of Artificial Sequence Synthetic Peptide
VTKAQKKDGKKRKRSRKESYSVYVYKVLKQ

SEQ ID NO: 78 - protein, 27 aa, Artificial Sequence
  Description of Artificial Sequence Synthetic Peptide
AQKKDGKKRKRSRKESYSVYVYKVLKQ

SEQ ID NO: 79 - protein, 24 aa, Artificial Sequence
  Description of Artificial Sequence Synthetic Peptide
KDGKKRKRSRKESYSVYVYKVLKQ

SEQ ID NO: 80 - protein, 21 aa, Artificial Sequence
  Description of Artificial Sequence Synthetic Peptide
KKRKRSRKESYSVYVYKVLKQ

SEQ ID NO: 81 - protein, 18 aa, Artificial Sequence
  Description of Artificial Sequence Synthetic Peptide
KRSRKESYSVYVYKVLKQ

SEQ ID NO: 82 - protein, 15 aa, Artificial Sequence
  Description of Artificial Sequence Synthetic Peptide
RKESYSVYVYKVLKQ

SEQ ID NO: 83 - protein, 12 aa, Artificial Sequence
  Description of Artificial Sequence Synthetic Peptide
SYSVYVYKVLKQ

SEQ ID NO: 84 - protein, 9 aa, Artificial Sequence
  Description of Artificial Sequence Synthetic Peptide
VYVYKVLKQ

SEQ ID NO: 85 - protein, 6 aa, Artificial Sequence
  Description of Artificial Sequence Synthetic Peptide
YKVLKQ

SEQ ID NO: 86 - protein, 5 aa, Artificial Sequence
  Description of Artificial Sequence Synthetic Peptide
KVLKQ

SEQ ID NO: 87 - protein, 25 aa, Artificial Sequence
  Description of Artificial Sequence Synthetic Peptide
RSVCRQIKICRRRGGCYYKCTNRPY

SEQ ID NO: 88 - protein, 33 aa, Artificial Sequence
  Description of Artificial Sequence Synthetic Peptide
GFFALIPKIISSPLFKTLLSAVGSALSSSGDQE

SEQ ID NO: 89 - protein, 24 aa, Artificial Sequence
  Description of Artificial Sequence Synthetic Peptide
GTAMRILGGVIPRKKRRQRRRPPQ

SEQ ID NO: 90 - protein, 25 aa, Artificial Sequence
  Description of Artificial Sequence Synthetic Peptide
KKKKKRFSFKKSFKLSGFSFKKNKK

SEQ ID NO: 91 - protein, 16 aa, Artificial Sequence
  Description of Artificial Sequence Synthetic Peptide
RQIKIWFQNRRMKWKK

SEQ ID NO: 92 - protein, 16 aa, Artificial Sequence
  Description of Artificial Sequence Synthetic Peptide
RQIRIWFQNRRMRWRR

SEQ ID NO: 93 - protein, 41 aa, Artificial Sequence
  Description of Artificial Sequence Synthetic Peptide
RKKRRQRRRPPVAYISRGGVSTYYSDTVKGRFTRQKYNKRA

SEQ ID NO: 94 - protein, 30 aa, Artificial Sequence
  Description of Artificial Sequence Synthetic Peptide
LGLLLRHLRHHSNLLANIPRKKRRQRRRPP

SEQ ID NO: 95 - protein, 21 aa, Artificial Sequence
  Description of Artificial Sequence Synthetic Peptide
KETWWETWWTEWSQPKKKRKV

SEQ ID NO: 96 - RNA, 21 nt, Artificial Sequence
  Description of Artificial Sequence Synthetic siRNA
uagggucggaacccaagcuua

(19) United States
(12) Patent Application Publication
(10) Pub. No.: US 2013/0072424 A1
(43) Pub. Date: Mar. 21, 2013

(54) COMPOUNDS AND METHODS FOR PEPTIDE RIBONUCLEIC ACID CONDENSATE PARTICLES FOR RNA THERAPEUTICS

(71) Applicants: Roger C. Adami, Bothell, WA (US); Tianying Zhu, Kirkland, WA (US); Kunyuan Cui, Bothell, WA (US); Michael E. Houston, JR., Sammamish, WA (US); Lishan Chen, Bellevue, WA (US); Yuching Chen, Bellevue, WA (US)

(72) Inventors: Roger C. Adami, Bothell, WA (US); Tianying Zhu, Kirkland, WA (US); Kunyuan Cui, Bothell, WA (US); Michael E. Houston, JR., Sammamish, WA (US); Lishan Chen, Bellevue, WA (US); Yuching Chen, Bellevue, WA (US)

(73) Assignee: MARINA BIOTECH, INC., Bothell, WA (US)

(21) Appl. No.: 13/475,892

(22) Filed: May 18, 2012

Related U.S. Application Data
(63) Continuation of application No. 12/445,575, filed on Apr. 14, 2009, now abandoned.
(60) Provisional application No. 60/727,216, filed on Oct. 14, 2005.
(60) Provisional application No. 60/733,664, filed on Nov. 4, 2005.
(60) Provisional application No. 60/825,878, filed on Sep. 15, 2006.

(51) Int. Cl. C07K 14/00; A61K 9/14

(52) U.S. Cl. CPC C07K 14/003; A61K 9/14
USPC 514/3.7; 530/322; 428/402

(57) ABSTRACT

Compounds comprising condensed particles having diameters less than 1000 nm, wherein the particles comprise one or more double stranded ribonucleic acids (dsRNAs) and one or more peptides. The compounds, compositions and methods are useful for modulating gene expression by RNA interference.

### FIELD OF THE INVENTION

This invention relates generally to the fields of RNA Interference, and delivery of RNA therapeutics. More particularly, this invention relates to compounds and compositions of peptide ribonucleic acid condensate particles, and their uses for medicaments and for delivery as therapeutics. This invention relates generally to methods of using peptide ribonucleic acid condensate compounds in RNA Interference for gene-specific inhibition of gene expression in mammals.

### BACKGROUND OF THE INVENTION

RNA Interference (RNAi) refers to methods of sequence-specific post-transcriptional gene silencing which is mediated by a double-stranded RNA (dsRNA) called a short interfering RNA (siRNA). See Fire, et al., Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When a siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

The development of RNAi therapy, antisense therapy, and gene therapy, among others, has created a need for effective means of introducing active nucleic acid-based agents into cells. In general, nucleic acids are stable for only very limited times in cells or plasma. However, nucleic acid-based agents can be stabilized by aggregation and binding into condensed compounds which may exhibit particles small enough for cellular delivery.

What is need are compounds comprised of small particles which contain an active nucleic acid agent for intracellular delivery and, ultimately, as a therapeutic, and methods for making such compounds. In particular, there is a need for compounds and methods to deliver double-stranded RNA to cells to produce the response of RNAi.

### SUMMARY OF THE INVENTION

This invention overcomes these and other drawbacks in the field by providing a range of peptide-ribonucleic acid compounds and compositions for use in RNA Interference and other therapeutic methods. This invention particularly provides compounds and methods of making compounds comprising one or more ribonucleic acid agents condensed with one or more peptides into small stable particles which are active to inhibit expression of targeted genes through RNA Interference. This summary, taken along with the description of drawings, detailed description of the invention, as well as the appended examples, claims, and drawings, as a whole, encompasses the invention disclosed.

In some aspects, this invention provides a range of peptide-RNA compounds and compositions for use in RNA Interference and other therapeutic methods, including compounds containing RNAs and peptides condensed into small, stable particles, which are active to inhibit expression of targeted genes through RNAi. The compounds of this invention are generally provided as a wide range of admixtures or condensates of synthetic peptides with nucleic acids.

In other aspects, the condensate compounds and compositions of this invention include small, stable particles of a peptide-RNA complex. In some embodiments, these compounds and particles can be further stabilized by crosslinking. In other embodiments, the compounds and compositions of this invention include a stealthing or surface modifying agent such as polyethylene glycol to enhance delivery.

In further aspects, the compounds of this invention include condensate complexes of one or more ribonucleic acids and one or more peptide components. The peptide components can have sufficient positive charge to bind to a ribonucleic acid to form a non-covalently linked peptide-ribonucleic acid condensate compound.

In some aspects, condensate compounds of this invention may form uniform particles. In some embodiments, the diameters of spherical particles of peptide-nucleic acid compounds may have a narrow distribution with an average of less than 1000 nanometers (nm).

The peptide-nucleic acid condensate compounds of this invention can provide their own multicomponent formulations. In some embodiments, a compound can be combined with other agents for drug delivery such as carriers or vehicles for delivery to a cell, or various delivery matrices, for in vivo therapeutics.

In some embodiments, compounds are provided from one or more ribonucleic acids and one or more peptides by dissolving at least one ribonucleic acid agent in an aqueous solution, then adding at least one peptide component to the aqueous solution thereby condensing particles having diameters less than 1000 nm, thereafter adding a second or successive peptide components to the aqueous solution, which adds mass to the particles.

In further embodiments, compounds are provided from one or more ribonucleic acid agents and one or more peptide components by dissolving a first peptide component in an aqueous solution, then adding the ribonucleic acid agent to the aqueous solution thereby condensing particles having diameters less than 1000 nm, thereafter adding a second or successive peptide components to the aqueous solution, which adds mass to the particles.

In one aspect of this invention, a peptide component is selected by its relative affinity for a nucleic acid. The peptide components can be selected to allow a variation of the degree of binding of the peptide components to the nucleic acids.

In some aspects, ribonucleic acid-peptide condensate compounds can be reversibly-bound. Compounds of ribonucleic acids and an amount of positively-charged ribonucleic acid-binding peptides can be substantially stable in an extracellular biological environment and release ribonucleic acids upon contact with an intracellular endosome. The release may produce the response of RNAi.

In further aspects, structures and methods of stabilizing a peptide-ribonucleic acid compound are provided including crosslinking ribonucleic acid-binding peptides within the compound. Methods of protecting a peptide-ribonucleic acid compound from degradation within a biological organism include crosslinking at least a portion of the peptides within the compound.

This invention further provides uses of the compounds as medicaments and in the manufacture of medicaments for use in RNAi therapy in animals and humans.

### BRIEF DESCRIPTION OF THE DRAWINGS

FIG. 1: Diameters of condensate particles of siRNA G1498 and peptide PN183 at various concentrations of G1498 and various nitrogen to phosphorous ratios (N/P). For each group of three bars at a particular N/P, the concentration of G1498 for the leftmost bar was 100 ug/ml, for the middle bar was 50 ug/ml, and for the rightmost bar was 10 ug/ml. At N/P of 0.2 and 0.5, the particles were very small when the concentration of G1498 was 10 ug/ml.

FIG. 2: Diameters of condensate particles of siRNA G1498 and peptide PN183 at various nitrogen to phosphorous ratios (N/P). For each group of two bars at a particular N/P, the left bar was with vortexing, while the right bar was without vortexing. Data obtained immediately after mixing.

FIG. 3: Diameters of condensate particles of siRNA G1498 and peptide PN183 at various nitrogen to phosphorous ratios (N/P). For each group of two bars at a particular N/P, the left bar was with vortexing, while the right bar was without vortexing. Data obtained 30 minutes after mixing.

FIG. 4: Diameters of condensate particles of siRNA G1498 and peptide PN183 at various nitrogen to phosphorous ratios (N/P). For each group of two bars at a particular N/P, the left bar was with vortexing, while the right bar was without vortexing. Data obtained 60 minutes after mixing.

FIG. 5: Diameters of condensate particles of siRNA G1498 and peptide PN183 at various nitrogen to phosphorous ratios (N/P). For each group of two bars at a particular N/P, the left bar was with vortexing, while the right bar was without vortexing. Data obtained 24 hours after mixing.

FIG. 6: Diameters of condensate particles of siRNA G1498 and peptide PN183 obtained at a concentration of G1498 of 100 ug/ml for various values of pH.

FIG. 7: Diameters of condensate particles of siRNA G1498 and peptide PN183 obtained as the concentration of sodium chloride was increased.

FIG. 8: Diameters of condensate particles of siRNA G1498 and peptide PN183 obtained at various N/P ratios and various order of addition of the components. For each group of two bars at a particular N/P, the left bar was obtained by adding siRNA first, while the right bar was obtained by adding the peptide first.

FIG. 9: Transmission electron micrograph of condensate particles of siRNA G1498 and peptide PN183. Length legend marker is 200 nm.

FIG. 10: Transmission electron micrograph of condensate particles of siRNA G1498 and peptide PN183. Length legend marker is 200 nm.

FIG. 11: Knockdown assay of LPS-induced TFN-α expression (pg/ml) in a mouse model by intranasal administration of a composition including condensate particles of siRNA Inm-4 and peptides PN183 and PN939. Buffer control is the leftmost bar, followed by data for condensate Inm-4/PN183/PN939, followed on the right by data for compound Inm-4/PN183/PN939 crosslinked with glutaraldehyde (G). Placebo does not contain the siRNA, and Qneg contains a non-active-siRNA.

FIG. 12: Knockdown in vitro assay of lac-z expression in rat gliosarcoma fibroblast cells 9 L/LacZ for condensates of the lac-z siRNA with peptide PN183 and various second peptides. Comparative data using HiPerFect™ (Qiagen; Valencia, Calif.) is the leftmost bar, followed by data for various compounds of this invention. The N/P ratio for PN183 was 0.75, while the N/P ratio for the second peptide was 0.3.

### DETAILED DESCRIPTION OF INVENTION

This invention provides a range of peptide-RNA compounds and compositions for use in RNA Interference and other therapeutic methods. More particularly, this invention includes compounds containing RNA and peptide condensed into small, stable particles, which are active to inhibit expression of targeted genes through RNAi.

The compounds of this invention are generally provided as admixtures or condensates of synthetic peptides with nucleic acids. A wide range of peptides may be used to form the compounds. The mass of a peptide is typically less than about 120 kDa, or less than about 60 kDa, or less than about 30 kDa. A peptide of the compound may be a mucosal permeability modulator or mucosal permeation enhancer.

The condensate compounds include small, stable particles of a peptide-RNA complex. These compounds and particles can be further stabilized by crosslinking with various reagents. In some embodiments, the compounds and compositions of this invention include a stealthing or surface modifying agent such as polyethylene glycol to enhance delivery.

The compounds of this invention include condensate complexes comprised of one or more ribonucleic acids and one or more peptide components. The peptide components can have sufficient positive charge to bind to a ribonucleic acid to form a non-covalently linked peptide-ribonucleic acid condensate compound. Stable ribonucleic acid complexes are provided which are comprised of ribonucleic acids and an amount of ribonucleic acid-binding peptides effective to stabilize the ribonucleic acid under in vivo conditions. The binding of the components of the peptide-nucleic acid complex is due partly to ionic forces, and can involve various other interactions such as van der Waals forces or hydrogen bonding.

Peptide-nucleic acid condensate compounds of this invention can comprise uniform particles. The diameters of spherical particles of the peptide-nucleic acid compounds may have a narrow distribution with an average of less than 1000 nanometers (nm). The diameters of spherical particles may be less than 1000 nanometers, from about 0.5 to about 400 nanometers, from about 10 to about 300 nanometers, and from about 40 to about 100 nanometers. The magnitude of the zeta potential for stable particles can be greater than about 20 millivolts, or greater than about 30 millivolts.

As used herein, the term “uniform” means that a substantial portion of the particles of a compound have a narrow distribution of diameters. More than one distribution of diameters may occur in a compound of uniform particles. A narrow distribution of diameters corresponds to a peak in the particle size distribution chart which is based on the raw correlation coefficient versus time data of a particle sizer instrument. Preferably, a uniform compound has at least 30% of the particles in one narrow distribution of diameters.

The peptide-nucleic acid condensate compounds of this invention provide their own multicomponent formulations, and can be further combined with other agents for drug delivery such as carriers or vehicles for delivery to a cell, or various delivery matrices, for in vivo therapeutics.

The compound and compositions of this invention may be dispersed within a pharmaceutically acceptable medium, associated with a matrix, or associated with a carrier or vehicle for delivery to a cell or subject. A solution comprised of a dispersion of the compounds or particles of this invention can be provided for delivery as a therapeutic.

### Peptide Components

Peptide components suitable for the compounds of this invention may be synthetically or derived from natural or other sources.

The peptide components can contain from 2 to about 1000 amino acids in length; from 2 to about 600 amino acids in length; from 2 to about 60 amino acids in length; from 5 to about 30 amino acids in length; and from 5 to about 25 amino acids in length.

The peptide components may comprise a plurality of positive charges. For example, a peptide component may comprise from 1 to about 100 positive charges, from 5 to about 30 positive charges, and from 9 to about 15 positive charges. The positive charges of a peptide component can be provided by positively-charged lysine or arginine residues.

A wide range of peptides may be used to form the peptide-nucleic acids compounds. The mass of a peptide component is typically less than about 120 kDa, or less than about 60 kDa, or less than about 30 kDa. The peptide of the peptide component may optionally be conjugated, or derivatized with a polymer such as a polyalkyleneoxide, polyethyleneoxide, polypropyleneoxide, or combinations thereof. For example, the peptide components of the compounds of this invention may be covalently derivatized with polyethyleneglycol (PEG).

Functional domains of the polynucleotide delivery-enhancing polypeptides are useful for the ability to deliver siNAs into cells. These functional domains include membrane attachment, fusogenic and nucleotide binding regions. Membrane attachment describes the ability of the exemplary polynucleotide delivery-enhancing polypeptide to bind the cell membrane. The fusogenic character reflects an ability to detach from the cell membrane and enter the cytoplasm. The membrane attachment and fusogenic domains of the peptide are closely linked mechanistically (i.e., peptide's ability to enter the cell) and therefore may be difficult to differentiate experimentally. Lastly, the nucleotide binding describes the peptide's ability to bind nucleotides.

A peptide of the compound may contain structural features which are known to enhance delivery of a compound across a barrier, such as a mucosal barrier. Examples of delivery enhancing features include various protein transduction domains. A peptide component can be a mucosal permeability modulator.

Examples of protein transduction domains for polynucleotide delivery-enhancing polypeptides of the invention include:

| | |
|---|---|
| 1. | TAT protein transduction domain (PTD) |
| | (SEQ ID NO: 1) |
| | KRRQRRR; |
| 2. | Penetratin PTD |
| | (SEQ ID NO: 2) |
| | RQIKIWFQNRRMKWKK; |
| 3. | VP22 PTD |
| | (SEQ ID NO: 3) |
| | DAATATRGRSAASRPTERPRAPARSASRPRRPVD; |
| 4. | Kaposi FGF signal sequences |
| | (SEQ ID NO: 4) |
| | AAVALLPAVLLALLAP, |
| | and |
| | SEQ ID NO: 5) |
| | AAVLLPVLLPVLLAAP; |
| 5. | Human β3 integrin signal sequence |
| | (SEQ ID NO: 6) |
| | VTVLALGALAGVGVG; |
| 6. | gp41 fusion sequence |
| | (SEQ ID NO: 7) |
| | GALFLGWLGAAGSTMGA; |
| 7. | Caiman crocodylus Ig(v) light chain |
| | (SEQ ID NO: 8) |
| | MGLGLHLLVLAAALQGA; |
| 8. | hCT-derived peptide |
| | (SEQ ID NO: 9) |
| | LGTYTQDFNKFHTFPQTAIGVGAP; |
| 9. | Transportan |
| | (SEQ ID NO: 10) |
| | GWTLNSAGYLLKINLKALAALAKKIL; |
| 10. | Loligomer |
| | (SEQ ID NO: 11) |
| | TPPKKKRKVEDPKKKK; |
| 11. | Arginine peptide |
| | (SEQ ID NO: 12) |
| | RRRRRRR; |
| | and |
| 12. | Amphiphilic model peptide |
| | (SEQ ID NO: 13) |
| | KLALKLALKALKAALKLA. |

Examples of viral fusion peptides fusogenic domains for polynucleotide delivery-enhancing polypeptides of this invention include:

| | |
|---|---|
| 1. | Influenza HA2 |
| | (SEQ ID NO: 14) |
| | GLFGAIAGFIENGWEG; |
| 2. | Sendai F1 |
| | (SEQ ID NO: 15) |
| | FFGAVIGTIALGVATA; |
| 3. | Respiratory Syncytial virus F1 |
| | (SEQ ID NO: 16) |
| | FLGFLLGVGSAIASGV; |
| 4. | HIV gp41 |
| | (SEQ ID NO: 17) |
| | GVFVLGFLGFLATAGS; |
| | and |
| 5. | Ebola GP2 |
| | (SEQ ID NO: 18) |
| | GAAIGLAWIPYFGPAA. |

In some embodiments, polynucleotide delivery-enhancing polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified in Table 1, below (see, e.g., Simpson, et al., J. Biol. Chem. 278:28011-28018, 2003).

In Table 1, the sequences for Sp1, Sp2, Sp3, Sp4, DrosBtd, DrosSp, CeT22C8.5, and Y4pB1A.4, are herein assigned SEQ ID NOS: 19, 20, 21, 22, 23, 24, 25, and 26, respectively.

Table 1 demonstrates a conservative zinc finger motif for double strand DNA binding which is characterized by the C-x(2,4)-C-x(12)-H-x(3)-H motif pattern (SEQ ID NO: 27), which itself can be used to select and design additional polynucleotide delivery-enhancing polypeptides according to the invention.

Alternative DNA binding domains useful for constructing polynucleotide delivery-enhancing polypeptides of this invention include, for example, portions of the HIV Tat protein sequence.

In some embodiments of this invention, polynucleotide delivery-enhancing polypeptides may be constructed by combining any of the foregoing structural elements, domains, or motifs into a single polypeptide which mediates enhanced delivery of siNAs into target cells. For example, a protein transduction domain of the TAT polypeptide may be fused to the N-terminal 20 amino acids of the influenza virus hemagglutinin protein, termed HA2, to yield a polynucleotide delivery-enhancing polypeptide.

The compounds of this invention can include one or more peptide components. A peptide component can have sufficient positive charge to bind a ribonucleic acid to form a non-covalently bound peptide-ribonucleic acid condensate compound. While the binding of the components of the peptide-nucleic acid complex is due partly to ionic forces, the binding can also involve various other interactions such as van der Waals forces, hydrogen bonding, or hydrophobic interactions. A complex may retain aqueous interactions, or a region of high solvent concentration.

Stable peptide-ribonucleic acid complexes are provided which comprise ribonucleic acids and an amount of ribonucleic acid-binding peptides effective to stabilize the ribonucleic acid under in vivo conditions.

Some example peptides useful for compounds of this invention are shown in Table 2.

TABLE 2
Peptides

| Peptide | Structure |
|---|---|
| PN183 | (SEQ ID NO: 28) |
| | NH₂-KETWWETWWTEWSQPGRKKRRQRRRPPQ- |
| | Amide |
| PN183 | (SEQ ID NO: 29) |
| Analog | NH₂-WWTWWWWWWWEWSQPKKKKRRRRRRPPQ- |
| 1 | Amide |
| PN183 | (SEQ ID NO: 30) |
| Analog | NH₂-WWWWWWWWWWSQPKKKKKKKKKK-Amide |
| 2 | |
| PN183 | (SEQ ID NO: 31) |
| Analog | NH₂-WWWWWWWWWWSQPRRRRRRRRRR-Amide |
| 3 | |
| PN183 | (SEQ ID NO: 32) |
| Analog | NH₂-KWWWWWWWWWEWSQPKKKKRRRRRRKKK- |
| 4 | Amide |
| PN938 | (SEQ ID NO: 33) |
| | NH₂-(Lys-His)₁₀-amide |
| PN939 | (SEQ ID NO: 34) |
| | PEG (2kSmall)-(Lys-His)₁₀-amide |
| PN951 | (SEQ ID NO: 35) |
| | NH₂-(His)₆-Arg-Ser-Val-Cys-Arg- |
| | Gln-Ile-Lys-Ile-Cys-Arg-Arg-Arg- |
| | Gly-Gly-Cys-Tyr-Try-Lys-Cys-Thr- |
| | Xaa-Arg-Pro-Tyr-amide |
| PN970 | (SEQ ID NO: 36) |
| | PEG (10kS.Mal)-(Lys)₉-Gly-Leu- |
| | Phe-Gly-Ala-Ile-Ala-Gly-Phe-Ile- |
| | Glu-Xaa-Gly-Trp-Glu-Gly-Met-Ile- |
| | Asp-Gly-amide |
| PN826 | (SEQ ID NO: 37) |
| | Ac- |
| | KGSKKAVTKAQKKEGKKRKRSRKESYSVYVYKVLKQ- |
| | amide |
| PN861 | (SEQ ID NO: 38) |
| | Ac-(Arg)₉-amide |
| PN924 | (SEQ ID NO: 39) |
| | NH2-(Lys)₂₀-amide |
| PN859 | (SEQ ID NO: 40) |
| | Ac-(Arg)₁₈-amide |
| PN907 | (SEQ ID NO: 41) |
| | PEG (10kS.Mal)-(Lys)₃₀-amide |
| PN73 | (SEQ ID NO: 42) |
| | NH2- |
| | KGSKKAVTKAQKKDGKKRKRSRKESYSVYVYKVLKQ- |
| | amide |
| PN526 | (SEQ ID NO: 43) |
| | PEG1-KLALKLALKALKAALKLA-amide |

Further examples of peptides useful for compounds of this invention are given in the examples appended below.

Condensate Compounds and their Preparation

This invention provides peptide-ribonucleic acid condensate compounds which can be comprised of particles having diameters less than about 1000 nm, from about 0.5 nm to about 400 nm; from about 10 nm to about 300 nm; and from about 40 nm to about 100 nm.

The peptide components of the compounds may be from 5-95% of the mass of the particles, or from 45-95% of the mass of the particles.

In some embodiments of this invention, peptide-nucleic acid compounds are provided from one or more ribonucleic acid agents and one or more peptide components by condensing the ribonucleic acid agents with the peptide components in an aqueous solution, thereby forming particles having diameters less than 1000 nm.

In general, the compounds of this invention comprise peptide-nucleic acid condensates having been formed from one or more peptides and one or more nucleic acids. The condensates are characterized in part by the nitrogen to phosphorous ratio (N/P ratio) for the peptides in relation to the nucleic acids.

A compound of this invention may be comprised of condensed particles having diameters less than 1000 nm, wherein each particle comprises at least 10 double stranded ribonucleic acid (dsRNA) molecules and at least 10 peptides. As used herein, “at least 10 peptides” refers to a partial molar quantity being 10 peptide molecules, which may be the same or different in structure. Thus, “at least 10 peptides” can be a partial molar quantity of a single peptide structure, or partial molar quantities of two or more different peptide structures.

In general, as used herein, terms such as “peptide” and “nucleic acid” and “dsRNA” and “siRNA” refer to an amount of those molecules sufficient to form a compound of this invention. In other words, in general, such terms refer to partial molar quantities rather than individual molecules. A “peptide” is one or more peptide molecules such as, for example, Avagadro's number of peptide molecules. “Adding two peptides to a ribonucleic acid agent” refers to an admixture of peptides of two different structures, each in partial molar quantity, to the ribonucleic acid agent.

The amount of peptide bound to the nucleic acids (NAs) in a complex or condensate can be obtained from the amount of bound nucleic acids using the peptide:NA charge ratio for single molecule pairing, also called the nitrogen to phosphorous ratio (N/P ratio). The amount of free peptide remaining in solution after condensation is given by mass balance. Thus, the charge ratio N/P herein refers to the initial charge ratio N/P of a single peptide component to a single nucleic acid agent in the initial condensate solution.

In general, the concentration of the nucleic acid agents in the solution is limited only by their solubility. The concentrations of the peptide components of the solution are adjusted to provide a desired N/P ratio.

In some embodiments, the concentrations of the peptide components of the solution are adjusted to provide a combined N/P ratio of about one. When the N/P ratio is about one, then on the basis of ionic charge neither the peptide components nor the nucleic acid agents are in excess.

In some embodiments, the concentration of each peptide component of the solution is adjusted to provide an N/P ratio of from about 0.2 to about 50, from about 0.5 to about 20, from about 0.5 to about 7, or from about 0.5 to about 2.5.

The pH of the solution is typically less than about 11, less than about 9, and less than about 8. The solution can optionally be vortexed for mixing the components.

In some embodiments, the condensate compounds are prepared by adding nucleic acid agents to a solution containing the peptide components.

In some embodiments, the solution may contain an inorganic or organic salt. For example, the aqueous solution may contain sodium chloride at a concentration of less than or equal to about 1 M, less than or equal to about 0.5 M, and less than or equal to about 0.25 M.

Optionally, the peptide-nucleic acid condensate compounds of a particular distribution of sizes can be isolated from the solution. In some embodiments, the solution containing the peptide-nucleic acid condensate compounds is filtered to isolate particles of various sizes.

In other embodiments, the solution containing the peptide-nucleic acid condensate compounds is dialyzed to remove excess or unbound peptide components.

In some embodiments, isolated peptide-nucleic acid particles are lyophilized.

In some embodiments of this invention, peptide-nucleic acid compounds are provided from one or more ribonucleic acid agents and one or more peptide components by dissolving at least one ribonucleic acid agent in an aqueous solution, then adding at least one peptide component to the aqueous solution thereby condensing particles having diameters less than 1000 nm, thereafter adding a second or successive peptide components to the aqueous solution, thereby adding mass to the particles.

In some embodiments of this invention, peptide-nucleic acid compounds are provided from one or more ribonucleic acid agents and one or more peptide components by dissolving a first peptide component in an aqueous solution, then adding the ribonucleic acid agent to the aqueous solution thereby condensing particles having diameters less than 1000 nm, thereafter adding a second or successive peptide components to the aqueous solution, thereby adding mass to the particles.

In one aspect of this invention, peptide-nucleic acid compounds are provided in which a peptide component is selected by its relative affinity for the nucleic acid. For example, a relative binding analysis of various peptides to a nucleic acid is performed by measurement of the displacement of SYBR-gold nucleic acid binding dye by the peptide. By characterizing the relative affinity of the peptide components for the nucleic acids of the compounds, the peptide components can be selected to allow a variation of the degree of binding of the peptide components to the nucleic acids.

Varying the degree of binding of the peptide components to the nucleic acids allows the condensate particles to be formed with a stronger-binding peptide component first, followed by a weaker-binding peptide component, or vice-versa, or to have multiple additions of components of variable binding strength.

In some embodiments, it is desirable to have the first peptide component which is condensed with the nucleic acid agent to have a higher binding affinity for the nucleic acid agent than succeeding peptide components. In these embodiments the concentration of the first peptide component of the solution is adjusted to provide an N/P ratio of from about 0.2 to about 7, from about 0.2 to about 2.5, or from about 0.2 to about 1. In these embodiments the concentrations of succeeding peptide components is adjusted to provide an NIP ratio of from about 0.2 to about 50, from about 0.5 to about 20, from about 0.5 to about 7, or from about 0.5 to about 2.5.

Reversibly-bound ribonucleic acid-peptide condensate compounds comprise ribonucleic acids and an amount of positively-charged ribonucleic acid-binding peptides that form a ribonucleic acid-peptide condensate that is substantially stable in an extracellular biological environment and that can release ribonucleic acids upon contact with an intracellular endosome.

A population of peptide-nucleic acid condensates is provided in which the peptides comprise an amount of positively-charged residues effective to bind ribonucleic acids. The ribonucleic acid-peptide condensates are substantially stable in an extracellular biological environment and can release ribonucleic acids intracellularly in a manner effective to produce the response of RNAi.

In some aspects of this invention, reagents are used to crosslink the peptide-RNA condensates. For example, the stability of peptide-RNA condensates may be increased by introducing dialdehyde groups, such as glutaraldehyde, to crosslink surface amine groups on the peptides or particles. Other examples of crosslinkers include formaldehyde, acrolein, and dithiobis(succinimidylpropionate). Crosslinked condensate compounds may have improved resistance to metabolism by serum endonucleases.

In some embodiments, a first peptide component which is condensed with the nucleic acid agent is crosslinked before the addition of successive peptide components. Optionally, the condensate of a first peptide component can be crosslinked after the addition of successive peptide components. In some embodiments, the condensate of a first peptide component is crosslinked before and after the addition of successive peptide components.

Methods of stabilizing a peptide-ribonucleic acid compound include crosslinking ribonucleic acid-binding peptides within the compound with, for example, a glutaraldehyde crosslinker. Methods of protecting a peptide-ribonucleic acid compound from degradation within a biological organism include crosslinking at least a portion of the peptides within the compound using, for example, a glutaraldehyde crosslinker.

The peptide-ribonucleic acid compounds of this invention can also be stabilized by addition of surface modifying agents such as surfactants, neutral lipids, or a polyethyleneoxide. For example, polyethylene glycol added to a solution of the condensate compounds can adhere to the particles thereof. A nonionic polyoxyethylene-polyoxypropylene block co-polymer may be added, for example, to stabilize the particles of the compound.

Uses of the compounds of this invention in the manufacture of medicaments for use in RNAi therapy in animals and humans are encompassed herein.

### Nucleic Acid Agents

Nucleic acid agents useful for this invention may be single-stranded nucleic acids, double-stranded nucleic acids, modified or degradation-resistant nucleic acids, RNA, a DNA-RNA chimera, an antisense nucleic acid, or a ribozyme.

In this context, this invention provides compounds, compositions and methods for modulating gene expression by RNA Interference. A compound or composition of this invention may release a ribonucleic acid agent to a cell which can produce the response of RNAi. Compounds or compositions of this invention may release ribonucleic acid agents to a cell upon contact with an intracellular endosome. The release of a ribonucleic acid agent intracellularly may provide inhibition of gene expression in the cell.

Ribonucleic acid agents useful for this invention may be targeted to various genes. For example, a siRNA agent of this invention may have a sequence that is complementary to a region of a TNF-alpha gene. In some embodiments of this invention, compounds and compositions are useful to regulate expression of tumor necrosis factor-α (TNF-α). TNF-α can be linked, for example, to inflammatory processes which occur in pulmonary diseases, and can have anti-inflammatory effects. Blocking TNF-α by delivery of a composition of this invention can be useful to treat or prevent the signs and/or symptoms of rheumatoid arthritis.

This invention provides compounds, compositions and methods for modulating expression and activity of TNF-α by RNA Interference.

Expression and/or activity of TNF-α can be modulated by delivering to a cell, for example, the siRNA molecule Inm-4. Inm-4 is a double stranded 21-nt siRNA molecule with sequence homology to the human TNF-α gene. Inm-4 has a 3′ dTdT overhang on the sense strand and a 3′ dAdT overhang on the antisense strand. The primary structure of Inm-4 is

sense
(SEQ ID NO: 44)
5′-CCGUCAGCCGAUUUGCUAUdTdT
antisense
(SEQ ID NO: 45)
5′-AUAGCAAAUCGGCUGACGGdTdT

Expression and/or activity of TNF-α can be modulated by delivering to a cell, for example, the siRNA molecule LC20. LC20 is a double stranded 21-nt siRNA molecule with sequence homology to the human TNF-α gene. LC20 is directed against the 3′-UTR region of human TNF-α. LC 20 has 19 base pairs with a 3′ dTdT overhang on the sense strand and a 3′ dAdT overhang on the antisense strand. The molecular weight of the sodium salt form is 14,298. The primary structure of LC20 is

sense
(SEQ ID NO: 46)
(5′) GGGUCGGAACCCAAGCUUAdTdT
antisense
(SEQ ID NO: 47)
(5′) UAAGCUUGGGUUCCGACCCdTdA

A siRNA of this invention may have a sequence that is complementary to a region of a viral gene. For example, some compositions and methods of this invention are useful to regulate expression of the viral genome of an influenza.

In this context, this invention provides compositions and methods for modulating expression and infectious activity of an influenza by RNA Interference. Expression and/or activity of an influenza can be modulated by delivering to a cell, for example, a short interfering RNA molecule having a sequence that is complementary to a region of a RNA polymerase subunit of an influenza. For example, in Table 3 are shown double-stranded siRNA molecules with sequence homology to an RNA polymerase subunit of an influenza.

TABLE 3
Double-Stranded siRNA
Molecules Targeted to Influenza

| siRNA | Subunit | SEQUENCE |
|---|---|---|
| G3789 | PB2 | (SEQ ID NO 48) |
| | | CGGGACUCUAGCAUACUUAdTdT |
| | | (SEQ ID NO 49) |
| | | UAAGUAUGCUAGAGUCCCGdTdT |
| G3807 | PB2 | (SEQ ID NO 50) |
| | | ACUGACAGCCAGACAGCGAdTdT |
| | | (SEQ ID NO 51) |
| | | UCGCUGUCUGGCUGUCAGUdTdT |
| G3817 | PB2 | (SEQ ID NO 52) |
| | | AGACAGCGACCAAAAGAAUdTdT |
| | | (SEQ ID NO 53) |
| | | AUUCUUUUGGUCGCUGUCUdTdT |
| G6124 | PB1 | (SEQ ID NO 54) |
| | | AUGAAGAUCUGUUCCACCAdTdT |
| | | (SEQ ID NO 55) |
| | | UGGUGGAACAGAUCUUCAUdTdT |
| G6129 | PB1 | (SEQ ID NO 56) |
| | | GAUCUGUUCCACCAUUGAAdTdT |
| | | (SEQ ID NO 57) |
| | | UUCAAUGGUGGAACAGAUCdTdT |
| G8282 | PA | (SEQ ID NO 58) |
| | | GCAAUUGAGGAGUGCCUGAdTdT |
| | | (SEQ ID NO 59) |
| | | UCAGGCACUCCUCAAUUGCdTdT |
| G8286 | PA | (SEQ ID NO 60) |
| | | UUGAGGAGUGCCUGAUUAAdTdT |
| | | (SEQ ID NO 61) |
| | | UUAAUCAGGCACUCCUCAAdTdT |
| G1498 | NP | (SEQ ID NO 62) |
| | | GGAUCUUAUUUCUUCGGAGdTdT |
| | | (SEQ ID NO 63) |
| | | CUCCGAAGAAAUAAGAUCCdTdT |

A siRNA of this invention may have a sequence that is complementary to a region of a RNA polymerase subunit of an influenza.

This invention provides compositions and methods to administer siNAs directed against a mRNA of an influenza, which effectively down-regulates an influenza RNA and thereby reduces, prevents, or ameliorates an influenza infection.

### RNA Interference Therapeutics

In some embodiments, this invention provides compounds, compositions and methods for inhibiting expression of a target transcript in a subject by administering to the subject a composition containing an effective amount of an RNAi-inducing compound such as a short interfering oligonucleotide molecule, or a precursor thereof. RNAi uses small interfering RNAs (siRNAs) to target messenger RNA (mRNAs) and attenuate translation. A siRNA as used in this invention may be a precursor for dicer processing such as, for example, a long dsRNA processed into a siRNA. This invention provides methods of treating or preventing diseases or conditions associated with expression of a target transcript or activity of a peptide or protein encoded by the target transcript.

A therapeutic strategy based on RNAi can be used to treat a wide range of diseases by shutting down the growth or function of a virus or microorganism, as well as by shutting down the function of an endogenous gene product in the pathway of the disease.

In some embodiments, this invention provides novel compositions and methods for delivery of RNAi-inducing compounds such as short interfering oligonucleotide molecules, and precursors thereof. In particular, this invention provides compositions containing an RNAi-inducing compound which is targeted to one or more transcripts of a cell, tissue, and/or organ of a subject.

A siRNA can be two RNA strands having a region of complementarity about 19 nucleotides in length. A siRNA optionally includes one or two single-stranded overhangs or loops.

A shRNA can be a single RNA strand having a region of self-complementarity. The single RNA strand may form a hairpin structure with a stem and loop and, optionally, one or more unpaired portions at the 5′ and/or 3′ portion of the RNA.

The active therapeutic agent can be a chemically-modified siNA with improved resistance to nuclease degradation in vivo, and/or improved cellular uptake, which retains RNAi activity.

A siRNA agent of this invention may have a sequence that is complementary to a region of a target gene. A siRNA of this invention may have 29-50 base pairs, for example, a dsRNA having a sequence that is complementary to a region of a target gene. Alternately, the double-stranded nucleic acid can be a dsDNA.

In some embodiments, the active agent can be a short interfering nucleic acid (siNA), short interfering RNA (siRNA), double-stranded RNA (dsRNA), micro-RNA, or short hairpin RNA (shRNA) that can modulate expression of a gene product.

Comparable methods and compositions are provided that target expression of one or more different genes associated with a particular disease condition in a subject, including any of a large number of genes whose expression is known to be aberrantly increased as a causal or contributing factor associated with the selected disease condition.

The RNAi-inducing compound of this invention can be administered in conjunction with other known treatments for a disease condition.

In some embodiments, this invention features compositions containing a small nucleic acid molecule, such as short interfering nucleic acid, a short interfering RNA, a double-stranded RNA, a micro-RNA, or a short hairpin RNA, admixed or complexed with, or conjugated to, a delivery-enhancing compound.

As used herein, the terms “short interfering nucleic acid,” “siNA,” “short interfering RNA,” “siRNA,” “short interfering nucleic acid molecule,” “short interfering oligonucleotide molecule,” and “chemically-modified short interfering nucleic acid molecule,” refer to any nucleic acid molecule capable of inhibiting or down regulating gene expression or viral replication, for example, by mediating RNA interference (RNAi) or gene silencing in a sequence-specific manner.

In some embodiments, the siNA is a double-stranded polynucleotide molecule comprising self-complementary sense and antisense regions, wherein the antisense region comprises a nucleotide sequence that is complementary to a nucleotide sequence in a target ribonucleic acid molecule for down regulating expression, or a portion thereof, and the sense region comprises a nucleotide sequence corresponding to (i.e., which is substantially identical in sequence to) the target ribonucleic acid sequence or portion thereof.

“siNA” means a small interfering nucleic acid, for example a siRNA, that is a short-length double-stranded nucleic acid, or optionally a longer precursor thereof. The length of useful siNAs within this invention will in some embodiments be optimized at a length of approximately 20 to 50 bp. However, there is no particular limitation to the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or after the time of delivery to yield a siNA that is active within the cell to mediate gene silencing. In some embodiments, useful siNAs will have a precursor length, for example, of approximately 100-200 base pairs, or 50-100 base pairs, or less than about 50 base pairs, which will yield an active, processed siNA within the target cell. In other embodiments, a useful siNA or siNA precursor will be approximately 10 to 49 bp, or 15 to 35 bp, or about 21 to 30 bp in length.

In some embodiments of this invention, polynucleotide delivery-enhancing polypeptides are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell.

For example, a siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

siNA molecules of this invention, particularly non-precursor forms, can be less than 30 base pairs, or about 17-19 bp, or 19-21 bp, or 21-23 bp.

siRNAs can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place within the region complementary to the antisense strand of the siRNA duplex. siRNA duplexes of 21 nucleotides are typically most active when containing two-nucleotide 3′-overhangs.

Replacing the 3′-overhanging segments of a 21-mer siRNA duplex having 2-nucleotide 3′ overhangs with deoxyribonucleotides may not have an adverse effect on RNAi activity. Replacing up to 4 nucleotides on each end of the siRNA with deoxyribonucleotides can be tolerated whereas complete substitution with deoxyribonucleotides may result in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long.

In some embodiments of this invention, the double-stranded region of siNAs in which two strands are paired may contain bulge or mismatched portions, or both. Double-stranded portions of siNAs in which two strands are paired are not limited to completely paired nucleotide segments, and may contain nonpairing portions due to, for example, mismatch (the corresponding nucleotides not being complementary), bulge (lacking in the corresponding complementary nucleotide on one strand), or overhang. Nonpairing portions can be contained to the extent that they do not interfere with siNA formation. In some embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other causes are also contemplated.

The terminal structure of siNAs of this invention may be either blunt or cohesive (overhanging) as long as the siNA retains its activity to silence expression of target genes. The cohesive (overhanging) end structure is not limited to the 3′ overhang, but includes the 5′ overhanging structure as long as it retains activity for inducing gene silencing. In addition, the number of overhanging nucleotides is not limited to 2 or 3 nucleotides, but can be any number of nucleotides as long as it retains activity for inducing gene silencing. For example, overhangs may comprise from 1 to about 8 nucleotides, or from 2 to 4 nucleotides.

The length of siNAs having cohesive (overhanging) end structure may be expressed in terms of the paired duplex portion and any overhanging portion at each end. For example, a 25/27-mer siNA duplex with a 2-bp 3′ antisense overhang has a 25-mer sense strand and a 27-mer antisense strand, where the paired portion has a length of 25 bp.

Any overhang sequence may have low specificity to a target gene, and may not be complementary (antisense) or identical (sense) to the target gene sequence. As long as the siNA retains activity for gene silencing, it may contain in the overhang portion a low molecular weight structure, for example, a natural RNA molecule such as a tRNA, an rRNA, a viral RNA, or an artificial RNA molecule.

The terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, or 15 to 35 bp, or about 21 to 30 bp long. Alternatively, the length of the double-stranded) region that is a final transcription product of siNAs to be expressed in a target cell may be, for example, approximately 15 to 49 bp, or 15 to 35 bp, or about 21 to 30 bp long.

The siNA can contain a single stranded polynucleotide having a nucleotide sequence complementary to a nucleotide sequence in a target nucleic acid molecule, or a portion thereof, wherein the single stranded polynucleotide can contain a terminal phosphate group, such as a 5′-phosphate (see for example, Martinez, et al., Cell. 110:563-574, 2002, and Schwarz, et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In some embodiments, the short interfering nucleic acid molecules of the invention lack 2′-hydroxy (2′-OH) containing nucleotides. In some embodiments, short interfering nucleic acids do not require the presence of nucleotides having a 2′-hydroxy group for mediating RNAi and as such, short interfering nucleic acid molecules of this invention optionally do not include any ribonucleotides (e.g., nucleotides having a 2′-OH group). siNA molecules that do not require the presence of ribonucleotides within the siNA molecule to support RNAi can, however, have an attached linker or linkers or other attached or associated groups, moieties, or chains containing one or more nucleotides with 2′-OH groups. siNA molecules can comprise ribonucleotides in at least about 5, 10, 20, 30, 40, or 50% of the nucleotide positions.

As used herein, the term siNA encompasses nucleic acid molecules that are capable of mediating sequence specific RNAi such as, for example, short interfering RNA (siRNA) molecules, double-stranded RNA (dsRNA) molecules, micro-RNA molecules, short hairpin RNA (shRNA) molecules, short interfering oligonucleotide molecules, short interfering nucleic acid molecules, short interfering modified oligonucleotide molecules, chemically-modified siRNA molecules, and post-transcriptional gene silencing RNA (ptgsRNA) molecules, among others.

In some embodiments, siNA molecules comprise separate sense and antisense sequences or regions, wherein the sense and antisense regions are covalently linked by nucleotide or non-nucleotide linker molecules, or are non-covalently linked by ionic interactions, hydrogen bonding, van der waals interactions, hydrophobic intercations, and/or stacking interactions.

“Antisense RNA” is an RNA strand having a sequence complementary to a target gene mRNA, that can induce RNAi by binding to the target gene mRNA.

“Sense RNA” is an RNA strand having a sequence complementary to an antisense RNA, and anneals to its complementary antisense RNA to form a siRNA.

As used herein, the term “RNAi construct” or “RNAi precursor” refers to an RNAi-inducing compound such as small interfering RNAs (siRNAs), hairpin RNAs, and other RNA species which can be cleaved in vivo to form a siRNA. RNAi precursors herein also include expression vectors (also referred to as RNAi expression vectors) capable of giving rise to transcripts which form dsRNAs or hairpin RNAs in cells, and/or transcripts which can produce siRNAs in vivo.

A siHybrid molecule is a double-stranded nucleic acid that has a similar function to siRNA. Instead of a double-stranded RNA molecule, a siHybrid is comprised of an RNA strand and a DNA strand. Preferably, the RNA strand is the antisense strand which binds to a target mRNA. The siHybrid created by the hybridization of the DNA and RNA strands have a hybridized complementary portion and preferably at least one 3′ overhanging end.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

In some embodiments, siNAs for intracellular delivery can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Examples of chemical modifications that can be made in an siNA include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation.

The antisense region of a siNA molecule can include a phosphorothioate internucleotide linkage at the 3′-end of said antisense region. The antisense region can comprise about one to about five phosphorothioate internucleotide linkages at the 5′-end of said antisense region. The 3′-terminal nucleotide overhangs of a siNA molecule can include ribonucleotides or deoxyribonucleotides that are chemically-modified at a nucleic acid sugar, base, or backbone. The 3′-terminal nucleotide overhangs can include one or more universal base ribonucleotides. The 3′-terminal nucleotide overhangs can comprise one or more acyclic nucleotides.

For example, a chemically-modified siNA can have 1, 2, 3, 4, 5, 6, 7, 8, or more phosphorothioate internucleotide linkages in one strand, or can have 1 to 8 or more phosphorothioate internucleotide linkages in each strand. The phosphorothioate internucleotide linkages can be present in one or both oligonucleotide strands of the siNA duplex, for example in the sense strand, the antisense strand, or both strands.

siNA molecules can comprise one or more phosphorothioate internucleotide linkages at the 3′-end, the 5′-end, or both of the 3′- and 5′-ends of the sense strand, the antisense strand, or in both strands. For example, an exemplary siNA molecule can include 1, 2, 3, 4, 5, or more consecutive phosphorothioate internucleotide linkages at the 5′-end of the sense strand, the antisense strand, or both strands.

In some embodiments, a siNA molecule includes 1, 2, 3, 4, 5, 6, 7, 8, 9, 10, or more pyrimidine phosphorothioate internucleotide linkages in the sense strand, the antisense strand, or in both strands.

In some embodiments, a siNA molecule includes 1, 2, 3, 4, 5, 6, 7, 8, 9, 10, or more purine phosphorothioate internucleotide linkages in the sense strand, the antisense strand, or in both strands.

A siNA molecule can include a circular nucleic acid molecule, wherein the siNA is about 38 to about 70, for example, about 38, 40, 45, 50, 55, 60, 65, or 70 nucleotides in length, having about 18 to about 23, for example, about 18, 19, 20, 21, 22, or 23 base pairs, wherein the circular oligonucleotide forms a dumbbell-shaped structure having about 19 base pairs and 2 loops.

A circular siNA molecule can contain two loop motifs, wherein one or both loop portions of the siNA molecule is biodegradable. For example, the loop portions of a circular siNA molecule may be transformed in vivo to generate a double-stranded siNA molecule with 3′-terminal overhangs, such as 3′-terminal nucleotide overhangs comprising about 2 nucleotides.

Modified nucleotides in a siNA molecule can be in the antisense strand, the sense strand, or both. For example, modified nucleotides can have a Northern conformation (e.g., Northern pseudorotation cycle, see for example, Saenger, Principles of Nucleic Acid Structure, Springer-Verlag ed., 1984). Examples of nucleotides having a Northern configuration include locked nucleic acid (LNA) nucleotides (e.g., 2′-O, 4′-C-methylene-(D-ribofuranosyl) nucleotides), 2′-methoxyethoxy (MOE) nucleotides, 2′-methyl-thio-ethyl, 2′-deoxy-2′-fluoro nucleotides, 2′-deoxy-2′-chloro nucleotides, 2′-azido nucleotides, and 2′-O-methyl nucleotides.

Chemically modified nucleotides can be resistant to nuclease degradation while at the same time maintaining the capacity to mediate RNAi.

The sense strand of a double stranded siNA molecule may have a terminal cap moiety such as an inverted deoxyabasic moiety, at the 3′-end, 5′-end, or both 3′ and 5′-ends of the sense strand.

Examples of conjugates include conjugates and ligands described in Vargeese, et al., U.S. application Ser. No. 10/427,160, filed Apr. 30, 2003, incorporated by reference herein in its entirety, including the drawings.

In some embodiments of this invention, the conjugate may be covalently attached to the chemically-modified siNA molecule via a biodegradable linker. For example, the conjugate molecule may be attached at the 3′-end of either the sense strand, the antisense strand, or both strands of the chemically-modified siNA molecule.

In some embodiments, the conjugate molecule is attached at the 5′-end of either the sense strand, the antisense strand, or both strands of the chemically-modified siNA molecule. In some embodiments, the conjugate molecule is attached both the 3′-end and 5′-end of either the sense strand, the antisense strand, or both strands of the chemically-modified siNA molecule, or any combination thereof.

In some embodiments, a conjugate molecule comprises a molecule that facilitates delivery of a chemically-modified siNA molecule into a biological system, such as a cell.

In some embodiments, a conjugate molecule attached to the chemically-modified siNA molecule is a polyethylene glycol, human serum albumin, or a ligand for a cellular receptor that can mediate cellular uptake. Examples of specific conjugate molecules contemplated by the instant invention that can be attached to chemically-modified siNA molecules are described in Vargeese, et al., U.S. Patent Publication No. 20030130186 and U.S. Patent Publication No. 20040110296, which are each hereby incorporated by reference in their entirety.

A siNA may be contain a nucleotide, non-nucleotide, or mixed nucleotide/non-nucleotide linker that joins the sense region of the siNA to the antisense region of the siNA. In some embodiments, a nucleotide linker can be 3, 4, 5, 6, 7, 8, 9, or 10 nucleotides in length. In some embodiments, the nucleotide linker can be a nucleic acid aptamer. As used herein, the terms “aptamer” or “nucleic acid aptamer” encompass a nucleic acid molecule that binds specifically to a target molecule, wherein the nucleic acid molecule contains a sequence that is recognized by the target molecule in its natural setting. Alternately, an aptamer can be a nucleic acid molecule that binds to a target molecule where the target molecule does not naturally bind to a nucleic acid.

For example, the aptamer can be used to bind to a ligand-binding domain of a protein, thereby preventing interaction of the naturally occurring ligand with the protein. See, for example, Gold, et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker can be an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993, and Biochemistry 32:1751, 1993; Durand, et al., Nucleic Acids Res. 18:6353, 1990; McCurdy, et al., Nucleosides & Nucleotides 10:287, 1991; Jschke, et al., Tetrahedron Lett. 34:301, 1993; Ono, et al., Biochemistry 30:9914, 1991; Arnold, et al., International Publication No. WO 89/02439; Usman, et al., International Publication No. WO 95/06731; Dudycz, et al., International Publication No. WO 95/11910, and Ferentz and Verdine, J. Am. Chem. Soc. 113:4000, 1991.

A “non-nucleotide linker” refers to a group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the Cl position of the sugar.

In some embodiments, modified siNA molecule can have phosphate backbone modifications including one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl substitutions. Examples of oligonucleotide backbone modifications are given in Hunziker and Leumann, Nucleic Acid Analogues: Synthesis and Properties, in Modern Synthetic Methods, VCH, pp. 331-417, 1995, and Mesmaeker, et al., Novel Backbone Replacements for Oligonucleotides, in Carbohydrate Modifications in Antisense Research, ACS, pp. 24-39, 1994.

siNA molecules, which can be chemically-modified, can be synthesized by: (a) synthesis of two complementary strands of the siNA molecule; and (b) annealing the two complementary strands together under conditions suitable to obtain a double-stranded siNA molecule. In some embodiments, synthesis of the complementary portions of the siNA molecule is by solid phase oligonucleotide synthesis, or by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,311. Synthesis of RNA, including certain siNA molecules of the invention, follows general procedures as described, for example, in Usman, et al., J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio. 74:59, 1997.

An “asymmetric hairpin” as used herein is a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop.

An “asymmetric duplex” as used herein is a siNA molecule having two separate strands comprising a sense region and an antisense region, wherein the sense region comprises fewer nucleotides than the antisense region to the extent that the sense region has enough complementary nucleotides to base pair with the antisense region and form a duplex.

To “modulate gene expression” as used herein is to upregulate or down-regulate expression of a target gene, which can include upregulation or downregulation of mRNA levels present in a cell, or of mRNA translation, or of synthesis of protein or protein subunits, encoded by the target gene.

The terms “inhibit”, “down-regulate”, or “reduce expression,” as used herein mean that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention.

“Gene silencing” as used herein refers to partial or complete inhibition of gene expression in a cell and may also be referred to as “gene knockdown.” The extent of gene silencing may be determined by methods known in the art, some of which are summarized in International Publication No. WO 99/32619.

As used herein, the terms “ribonucleic acid” and “RNA” refer to a molecule containing at least one ribonucleotide residue. A ribonucleotide is a nucleotide with a hydroxyl group at the 2′ position of a beta-D-ribo-furanose moiety. These terms include double-stranded RNA, single-stranded RNA, isolated RNA such as partially purified RNA, essentially pure RNA, synthetic RNA, recombinantly produced RNA, as well as modified and altered RNA that differs from naturally occurring RNA by the addition, deletion, substitution, modification, and/or alteration of one or more nucleotides. Alterations of an RNA can include addition of non-nucleotide material, such as to the end(s) of a siNA or internally, for example at one or more nucleotides of an RNA.

Nucleotides in an RNA molecule include non-standard nucleotides, such as non-naturally occurring nucleotides or chemically synthesized nucleotides or deoxynucleotides. These altered RNAs can be referred to as analogs.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can include a nucleic acid sequence having complementarity to a sense region of the siNA molecule.

By “target nucleic acid” is meant any nucleic acid sequence whose expression or activity is to be modulated. A target nucleic acid can be DNA or RNA.

By “complementarity” is meant that a nucleic acid can form hydrogen bond(s) with another nucleic acid sequence either by traditional Watson-Crick or by other non-traditional modes of binding.

The term “biodegradable linker” as used herein, refers to a nucleic acid or non-nucleic acid linker molecule that is designed as a biodegradable linker to connect one molecule to another molecule, for example, a biologically active molecule to a siNA molecule or the sense and antisense strands of a siNA molecule. The biodegradable linker is designed such that its stability can be modulated for a particular purpose, such as delivery to a particular tissue or cell type. The stability of a nucleic acid-based biodegradable linker molecule can be variously modulated, for example, by combinations of ribonucleotides, deoxyribonucleotides, and chemically-modified nucleotides, such as 2′-O-methyl, 2′-fluoro, 2′-amino, 2′-β-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

In connection with 2′-modified nucleotides as described herein, by “amino” is meant 2′-NH₂ or 2′-O—NH₂, which can be modified or unmodified. Such modified groups are described, for example, in Eckstein, et al., U.S. Pat. No. 5,672,695 and Matulic-Adamic, et al., U.S. Pat. No. 6,248,878.

### Administration

Some methods for delivery of nucleic acid molecules for use within then invention are described, for example, in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any nucleic acid molecule contemplated within the invention.

Nucleic acid molecules and peptides can be administered to cells by a variety of methods known to those of skill in the art, including, but not restricted to, administration within formulations that comprise the siNA and peptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the peptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Corry et al., Clin. Cancer Res. 5:2330-2337, 1999, and Barry, et al., International PCT Publication No: WO 99/31262.

The compositions of the instant invention can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments the invention provides pharmaceutical compositions and methods featuring the presence or administration of one or more polynucleic acid(s), typically one or more siNAs, combined, complexed, or conjugated with a peptide, optionally formulated with a pharmaceutically-acceptable carrier, such as a diluent, stabilizer, buffer, and the like.

The present invention satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of the invention may be targeted to lower expression of one gene, which can result in upregulation of a “downstream” gene whose expression is negatively regulated by a product or activity of the target gene.

This siNAs of the present invention may be administered in any form, for example transdermally or by local injection. Comparable methods and compositions are provided that target expression of one or more different genes associated with a selected disease condition in animal subjects, including any of a large number of genes whose expression is known to be aberrantly increased as a causal or contributing factor associated with the selected disease condition.

Negatively charged polynucleotides of the invention (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez, et al., Bioconjugate Chem. 10:1068-1074, 1999; Wang, et al., International PCT Publication Nos. WO 03/47518 and WO 03/46185), poly(lactic-co-glycolic acid) (PLGA) and PLCA microspheres (see for example, U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

Any one or combination of the cationic peptides of the present invention may be selected or combined to yield effective polynucleotide delivery-enhancing polypeptide reagents to induce or facilitate intracellular delivery of siNAs within the methods and compositions of the invention.

### Pharmaceutical Composition

The present invention also includes pharmaceutically acceptable formulations or compositions of the compounds described herein. These formulations include organic and inorganic salts of the above compounds, e.g., acid addition salts, for example, salts of hydrochloric, hydrobromic, acetic acid, and benzene sulfonic acid.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Additional excipients, for example sweetening, flavoring and coloring agents, can also be present.

Pharmaceutical compositions of this invention can also be in the form of oil-in-water emulsions. The oily phase can be a vegetable oil, or a mineral oil, or mixtures thereof. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

The pharmaceutical compositions can be in the form of a sterile injectable aqueous or oleaginous suspension. This suspension can be formulated using a suitable dispersing or wetting agent, and/or a suspending agent. A sterile injectable preparation can also be a sterile injectable solution or suspension in a non-toxic parentally acceptable diluent or solvent, for example as a solution in 1,3-butanediol.

Among the acceptable carriers, vehicles and solvents for a pharmaceutical composition that can be employed are water, Ringer's solution, and isotonic sodium chloride solution. In addition, sterile, fixed oils are conventionally employed as a carrier, vehicle, solvent, or suspending medium. For this purpose, any bland fixed oil can be employed including synthetic mono- or diglycerides. In addition, fatty acids such as oleic acid find use in the preparation of injectables.

All publications, references, patents, and patent applications cited herein are each hereby specifically incorporated by reference in their entirety.

While this invention has been described in relation to certain embodiments; and many details have been set forth for purposes of illustration, it will be apparent to those skilled in the art that this invention includes additional embodiments, and that some of the details described herein may be varied considerably without departing from this invention. This invention includes such additional embodiments, modifications and equivalents.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein is intended to refer individually to each separate value falling within the range as if it were individually recited herein, whether or not some of the values within the range are expressly recited. Specific values employed herein will be understood as exemplary and not to limit the scope of the invention.

The examples given herein, and the exemplary language used herein are solely for the purpose of illustration, and are not intended to limit the scope of the invention.

### EXAMPLES

### Preparation Example 1

PN0826:siRNA compounds in water. A compound was prepared by: Adding 82.12 μl of RNase free water to a centrifuge tube, and then 10 μl of G1498 (1 mg/ml, in RNase free water). The solution was vortexed to mix. Finally, 7.88 μl of PN0826 was added (1 mg/ml, in RNase free water) and vortexed to mix.

### Preparation Example 2

PN0826, F-108 and water. A compound was prepared by: in a centrifuge tube, 82.12 μl RNase free water was added first and then 10 μl of G1498 (1 mg/ml, in RNase free water). Vortexed to mix. Then added 7.88 μl of PN0826 (1 mg/ml, in RNase free water) and vortexed to mix. Finally added 5 μl Pluronic F108 (20 mg/ml, 0.2 μM filtered) and pipetted to mix.

### Preparation Example 3

Cy5-Inm4, PN0183 and overnight. A compound was prepared by: in a centrifuge tube, 119.40 μl of 10 mM Hepes/5% dextrose buffer (pH 5.0) was added first, then 15.60 μl of peptide PN0183 (2 mg/ml, in RNase free water), and vortexed to mix. The solution was stored at 4° overnight. Finally, 15 μl Cy5-Inm4 (1 mg/ml, in RNase free water) was added and vortexed again to mix.

### Preparation Example 4

Cy5-Inm4, PN0183, F127 and overnight. A compound was prepared by: in a centrifuge tube, 119.40 μl of 10 mM Hepes/5% dextrose buffer (pH 5.0) was added first, then 15.60 μl of peptide PN0183 (2 mg/ml, in RNase free water) and 7.5 μl Pluronic F127 (20 mg/ml, 0.2 μM filtered). Vortexed to mix. The solution was stored at 4° overnight. Finally, 15 μl Cy5-Inm4 (1 mg/ml, in RNase free water) was added and vortexed again to mix.

### Preparation Example 5

G1498, PN0183, water for dilution and peptide first. A compound was prepared by: in a centrifuge tube, 85.83 μl of 10 mM Hepes/5% dextrose buffer (pH5.0) was added first, then 4.17 μl of peptide PN0183 (5 mg/ml, in RNase free water), and vortexed to mix. Finally, 10 μl G1498 (1 mg/ml, in RNase free water) was added to the solution and vortexed again to mix.

### Preparation Example 6

G1498, PN0183, buffer for dilution and peptide first. A compound was prepared by: in a centrifuge tube, 85.83 μl of 10 mM Hepes/5% dextrose buffer (pH 5.0) was added first, then 4.17 μl of peptide PN0183 (5 mg/ml, in 10 mM Hepes/5% dextrose buffer of pH 5.0), and vortexed to mix. Finally, 10 μl G1498 (1 mg/ml, in 10 mM Hepes/5% dextrose buffer of pH 5.0) was added to the solution and vortexed again to mix.

### Preparation Example 7

G1498, PN0183, peptide first and without vortexing. A compound was prepared by: in a centrifuge tube, 85.83 μl of 10 mM Hepes/5% dextrose buffer (pH5.0) was added first, then 4.17 μl of peptide PN0183 (5 mg/ml, in 10 mM Hepes/5% dextrose buffer of pH 5.0), and pipetted to mix. Finally, 10 μl G1498 (1 mg/ml, in 10 mM Hepes/5% dextrose buffer of pH 5.0) was added to the solution and pipetted again to mix.

### Preparation Example 8

G1498, PN0183, peptide first and lower concentration by diluting down. A compound was prepared by: in a centrifuge tube, 85.83 μl of 10 mM Hepes/5% dextrose buffer (pH 5.0) was added first, then 4.17 μl of peptide PN0183 (5 mg/ml, in 10 mM Hepes/5% dextrose buffer of pH 5.0), and vortexed to mix. 10 μl G1498 (1 mg/ml, in 10 mM Hepes/5% dextrose buffer of pH 5.0) was added to the solution and vortexed again to mix. Finally, the solution was diluted 10 times to lower concentration.

### Preparation Example 9

G1498, PN0183 and siRNA first. A compound was prepared by: in a centrifuge tube, 85.83 μl of 10 mM Hepes/5% dextrose buffer (pH 5.0) was added first, then 10 μl G1498 (1 mg/ml, in 10 mM Hepes/5% dextrose buffer of pH 5.0) and vortexed to mix.

### Preparation Example 10

G1498, PN0183, peptide first and wait for 30 minutes. A compound was prepared by: in a centrifuge tube, 85.83 μl of 10 mM Hepes/5% dextrose buffer (pH 5.0) was added first, then 4.17 μl of peptide PN0183 (5 mg/ml, in 10 mM Hepes/5% dextrose buffer of pH 5.0), and vortexed to mix. Finally, 10 μl G1498 (1 mg/ml, in 10 M Hepes/5% dextrose buffer of pH 5.0) was added to the solution and vortexed again to mix. The solution was equilibrated on ice for 30 minutes.

### Preparation Example 11

G1498, PN0183, peptide first and wait for 60 minutes. A compound was prepared by: in a centrifuge tube, 85.83 μl of 10 mM Hepes/5% dextrose buffer (pH 5.0) was added first, then 4.17 μl of peptide PN0183 (5 mg/ml, in 10 mM Hepes/5% dextrose buffer of pH 5.0), and vortexed to mix. Finally, 10 μl G1498 (1 mg/ml, in 10 mM Hepes/5% dextrose buffer of pH 5.0) was added to the solution and vortexed again to mix. The solution was equilibrated on ice for 60 minutes.

### Preparation Example 12

G1498, PN0183, peptide first and wait for 24 hrs. A compound was prepared by: in a centrifuge tube, 85.83 μl of 10 mM Hepes/5% dextrose buffer (pH 5.0) was added first, then 4.17 μl of peptide PN0183 (5 mg/ml, in 10 mM Hepes/5% dextrose buffer of pH 5.0), and vortexed to mix. Finally, 10 μl G1498 (1 mg/ml, in 10 mM Hepes/5% dextrose buffer of pH 5.0) was added to the solution and vortexed again to mix. The solution was equilibrated on ice for 24 hrs.

### Preparation Example 13

Inm4, PN0183, PN0939 and siRNA added right before dosing. A compound was prepared by: in a centrifuge tube, 259.1 μl of 10 mM Hepes/5% dextrose buffer (pH 5.0) was added first, then 15.60 μl of PN0183 (5 mg/ml, in 10 mM Hepes/5% dextrose buffer of pH 5.0) and 10.30 μl of PN0939 (5 mg/ml, in 10 mM Hepes/5% dextrose buffer of pH 5.0). Vortexed to mix. Finally 15.00 μl of Inm4 (5 mg/ml, 10 mM Hepes/5% dextrose buffer of pH 5.0) was added. Vortexed to mix.

### Preparation Example 14

Inm4, order of siRNA, PN0183, PN0939 and pipetted to mix. A compound was prepared by: in a centrifuge tube, 172.00 of 10 mM Hepes/5% dextrose buffer (pH 5.0) was added first, then 10 μl Inm4 (5 mg/ml, in 10 mM Hepes/5% dextrose buffer of pH 5.0). Pipetted to mix. Later 11.20 μl of PN0183 (5 mg/ml, in 10 mM Hepes/5% dextrose buffer of pH 5.0) was added. Pipetted to mix. Finally added 6.80 μl of PN0939 (5 mg/ml, in 10 mM Hepes/5% dextrose buffer of pH 5.0). Pipetted again to mix. The solution was equilibrated on ice for 1 hr.

### Preparation Example 15

Inm4, order of siRNA, PN0183, PN0939 and vortexed to mix. A compound was prepared by: in a centrifuge tube, 2289.50 μl of 10 mM Hepes/5% dextrose buffer (pH 5.0) was added first, then 24.00 μl of Inm4 (20 mg/ml, in RNase free water). Vortexed to mix. Later 53.60 μl of PN0183 (10 mg/ml, in 10 mM Hepes/5% dextrose buffer of pH 5.0) was added. Vortexed to mix. Finally 32.90 μl of PN0939 (20 mg/ml, in 10 mM Hepes/5% dextrose buffer of pH 5.0) was added. Pipetted to mix. The solution was equilibrated on ice for 1 hr.

### Preparation Example 16

Inm4, order of siRNA, PN0183, PN0939 and pH 7.4. A compound was prepared by: in a centrifuge tube, 376.19 μl of 10 mM Hepes/5% dextrose buffer (pH 7.4) was added first, then 5 μl Inm4 (20 mg/ml, in RNase free water). Vortexed to mix. Later 15.39 μl PN0183 (7.26 mg/ml, in RNase free water) was added. Vortexed to mix. Finally 3.42 μl of PN0939 was added. Pipetted to mix.

### Preparation Example 17

G1498, PN0183 and tert-Butanol. A compound was prepared by: in a centrifuge tube, 72.93 μl of 10 mM Hepes/5% dextrose buffer (pH 5.0) was added first, then 4.17 μl PN0183 (5 mg/ml, in 10 mM Hepes/5% dextrose buffer of pH 5.0). Vortexed to mix. Later added 10 μl of G1498 (1 mg/ml, in 10 mM Hepes/5% dextrose buffer of pH 5.0). Vortexed again to mix. Finally, added 12.90 μl of Tert-Butanol and pipetted to mix.

### Preparation Example 18

G1498, PN0183 and ethanol. A compound was prepared: in a centrifuge tube, 73.33 μl of 10 mM Hepes/5% dextrose buffer (pH 5.0) was added first, then 4.17 μl PN0183 (5 mg/ml, in 10 mM Hepes/5% dextrose buffer of pH 5.0). Vortexed to mix. Later added 10 μl of G1498 (1 mg/ml, in 10 mM Hepes/5% dextrose buffer of pH 5.0). Vortexed again to mix. Finally, added 12.50 μl of Ethanol and pipetted to mix.

### Preparation Example 19

Lac-Z, PN0183, PN0939. A compound was prepared by: diluted 5.0 μl of Lac-Z siRNA (20 μM) into 120 μl OPTI-MEM medium. Added 1.62 μl PN0183 (1 mg/ml) and 1.98 μl PN0939 (1 mg/ml) into 121.40 μl. of OPTI-MEM medium. Combine the two solutions and pipetted to mix.

The structure of Lac-Z is:

Sense: CN2938.
(SEQ ID NO: 64)
5′-r(CUACACAAAUCAGCGAUUU)dTdT-3′
Antisense: CN2939.
(SEQ ID NO: 65)
5′-r(AAAUCGCUGAUUUGUGUAG)dTdC-3′

### Preparation Example 20

Lac-Z, PN0183, PN0938. A compound was prepared by: diluted 5.0 μl of Lac-Z siRNA (20 μM) into 120 μl OPTI-MEM medium. Added 1.62 μl PN0183 (1 mg/ml) and 0.97 μl PN0938 (1 mg/ml) together into 122.41 μl of OPTI-MEM medium. Combine the two solutions and pipetted to mix.

### Preparation Example 21

Lac-Z, PN0183, PN0939 and crosslinking. A compound was prepared by: Diluted 5.0 μl of Lac-Z siRNA (20 μM) into 120 μl OPTI-MEM medium. Added 1.62 μl PN0183 (1 mg/ml) and 1.98 μl PN0939 (1 mg/ml) into 119.80 μl of OPTI-MEM medium. Combined the two solutions and pipetted to mix. Then added 1.60 μl of Glutaraldehyde (0.05%, W/V) and pipetted to mix. The solution was equilibrated at room temperature for 1 hr.

### Preparation Example 22

Lac-Z, PN0183, crosslinking and PN0939. A compound was prepared: Diluted 5.0 μl of Lac-Z siRNA (20 μM) into 120 μl OPTI-MEM medium. Added 1.62 μl PN0183 (1 mg/ml) into 119.80 μl of OPTI-MEM medium. Combined the two solutions and then added 1.60 μl of Glutaraldehyde (0.05%, W/V). Pipetted to mix. This solution was equilibrated at room temperature for 1 hr. Finally, added 1.98 μl PN0939 (1 mg/ml). Pipetted to mix.

### Preparation Example 23

Lac-Z, PN0183, crosslinking, PN0939, and crosslinking. A compound was prepared by: Diluted 5.0 μl of Lac-Z siRNA (20 μM) into 120 μl OPTI-MEM medium. Added 1.62 μl PN0183 (1 mg/ml) into 119.80 μl of OPTI-MEM medium. Combined the two solutions and then added 0.8 μl of Glutaraldehyde (0.05%, W/V). Pipetted to mix. This solution was equilibrated at room temperature for 1 hr. And then added 1.98 μl PN0939 (1 mg/ml) and 0.8 μl of Glutaraldehyde (0.05%, W/V). Pipetted to mix.

### Preparation Example 24

Lac-Z, PN0183, crosslinking, dialysis and PN0939. A compound was prepared by: Made the Lac-Z siRNA and PN0183 combination first by adding 158.6 μl of 10 mM Hepes/5% dextrose buffer (pH 7.4), 103.45 μl of Lac-Z siRNA (20 μM) and 33.53 μl PN0183 (1 mg/ml). Vortexed to mix. Then added 4.4 μl of Glutaraldehyde (0.05%, W/V). Pipetted to mix. This solution was equilibrated at room temperature for 2 hrs. Then the solution was dialyzed at 4° for overnight. Diluted 43.5 μl of crosslinked combination into 331.5 μl of OPTI-MEM. Diluted 4.96 μl of PN0939 (0.1 mg/ml) into 57.54 μl of OPTI-MEM. Combine the two diluted solutions and pipetted to mix.

### Preparation Example 25

Lac-Z, PN0183, PN0826 and PEG 3350. A compound was prepared by: added 5.0 μl of Lac-Z siRNA (20 μM) and 1.6 μl PN0183 (0.1 mg/ml) into 120 μl OPTI-MEM medium and vortexed to mix. Added 3.96 μl PN0826 (0.1 mg/ml) and 2.50 μl of PEG 3350 (10 mg/ml) into 118.54 μl of OPTI-MEM medium. Combine the two solutions and pipetted to mix.

### Example 1

### Gold Dye Displacement Assay for Peptide-siRNA Affinity

The relative binding of various peptides to siRNA via a rapid screen was assessed by indirect measurement of the displacement of SYBR-gold nucleic acid binding dye. A buffered mixture of siRNA, peptide and SYBR-gold was prepared in the measurement plate in duplicate such that the peptide and SYBR-gold dye underwent simultaneous competitive binding of the siRNA. The concentration of siRNA was fixed at 10 pg/mL and was combined with a titration of each peptide ranging in a concentration that corresponded to a peptide:siRNA charge ratio between 0.05 and 10. Since SYBR-gold dye only fluoresces when bound to siRNA, peptide binding to the siRNA inhibits binding of the dye and consequently reduces the fluorescence. Therefore, the amount of fluorescence correlated inversely to the binding of the peptide to the siRNA. Both Kd and Bₘₐₓ values were calculated. A greater Kd value indicated greater binding affinity between the peptide and the siRNA.

SYBR-gold nucleic acid binding dye stock, a 10,000× concentrate, was supplied by Invitrogen (Carlsbad, Calif.) and stored at −20° C. The concentrate was allowed to equilibrate to room temperature before diluting 1 to 100 in Hyclone nuclease free water. This was diluted 1 to 10 in the experimental plate for a final concentrate of 10× for the assay. This was the optimal dilution to achieve linear binding to siRNA duplex at a concentration range of up to 50 pg/mL concentration. The values used to generate the standard curve demonstrating linear binding of SYBR-gold to G1498 siRNA are shown in Table 4.

TABLE 4
G1498 siRNA Standard Curve Values

| [G1498] | Mean | |
|---|---|---|
| μg/mL | Fluorescence | Std Dev |
| 0 | 0 | 3.86 |
| 1.56 | 376 | 10.0 |
| 3.13 | 840 | 44.8 |
| 6.25 | 3254 | 91.4 |
| 12.5 | 10591 | 762 |
| 25.0 | 26276 | 1497 |
| 50.0 | 36543 | 240 |

Samples were mixed directly in the 384 well analysis plate. First, 54, SYBR-gold dye was pipetted into each well with a multichannel pipet, touching the tip to the bottom of the well to draw out the solution completely. Second, 22.5 μL of 2× peptide solution was added with a single channel pipet. Finally, 22.5 μL of 2×siRNA was added with a multichannel pipet. The plate was covered immediately with foil and tapped gently to mix and draw down any droplets on the side of the well.

Fluorescence was measured using the SpectraMax fluorescent plate reader from Molecular Devices (Sunnyvale, Calif.). Plate settings included shaking before reading, one read per well, with excitation wavelength of 495 nm and emission wavelength of 537 nm. The plate was read within 30 minutes of the addition of the siRNA.

### Scatchard Plot for Peptide Binding

A Scatchard Plot is a plot of peptide binding ([peptide]bound/[peptide]free) vs. [peptide]bound. The slope of the linear regression of this plot is −1/Kd and Bmax is the y-intercept. Since the concentration of free and bound peptide cannot be measured directly, indirect measurement of siRNA was used for the calculation. Free siRNA was determined from measured fluorescence using the standard curve. Bound siRNA was determined from the standard curve by mass balance from the known initial siRNA concentration (10 μg/mL).

Bound peptide was calculated from bound siRNA by assuming the (siRNA:Peptide) bound molar ratio was equal to the (siRNA:Peptide) charge ratio for single molecule pairing. From this calculated bound peptide amount, the free peptide was calculated by mass balance.

### Particle Size and Zeta Potential

Particle size and zeta potential were determined with a Malvern Zetasizer Nano ZS (Malvern, Worcestershire, UK) using a DTS1060C clear disposable zeta cell at 25°. The dispersant for particle size was PBS, 1.0200 CP viscosity, or water, 0.8872 CP viscosity. The dispersant for zeta potential was water 0.8872 CP viscosity. The dispersant viscosity was used as the sample viscosity. When both the zeta potential and the particle size were measured, the clear disposable zeta cell was used. When only the particle size was measured, then a low volume disposable sizing cuvette was used.

### Example 2

### Condensate Particle Size at Various Nucleic Acid Concentrations and N/P Ratios

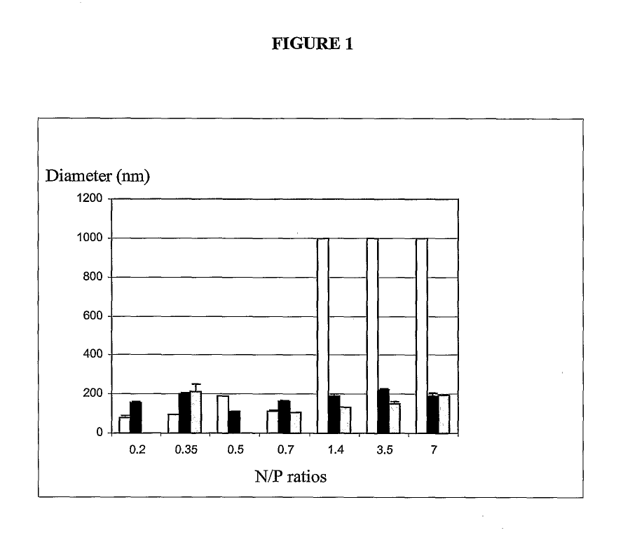

Diameters of particles of a condensate compound of siRNA G1498 and peptide PN183 at various concentrations of G1498 and various N/P ratios are shown in FIG. 1. For each group of three bars at a particular N/P, the concentration of G1498 for the leftmost bar was 100 ug/ml, for the middle bar was 50 ug/ml, and for the rightmost bar was 10 ug/ml. At N/P of 0.2 and 0.5, the particles were very small when the concentration of G1498 was 10 ug/ml, thus the bar does not appear.

At N/P ratios below about 1.4, the particle size was below about 200 nm for all concentrations of the siRNA. At N/P ratios at or above about 1.4, condensate particle size remained below about 200 nm for all concentrations of RNA except the highest (100 ug/ml).

### Example 3

### Condensate Particle Size at Various Nucleic Acid Concentrations and N/P Ratios

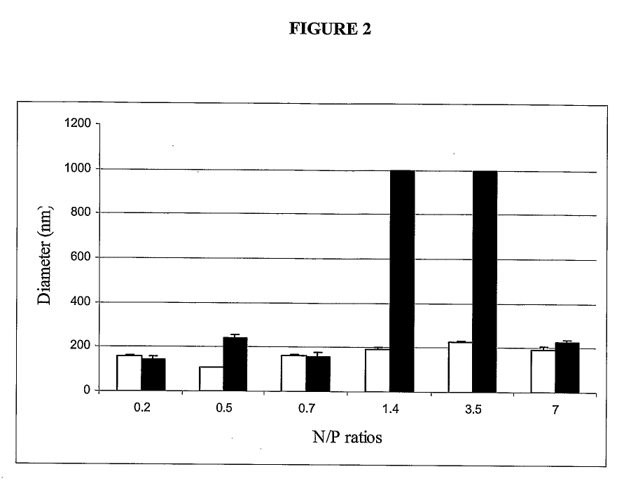

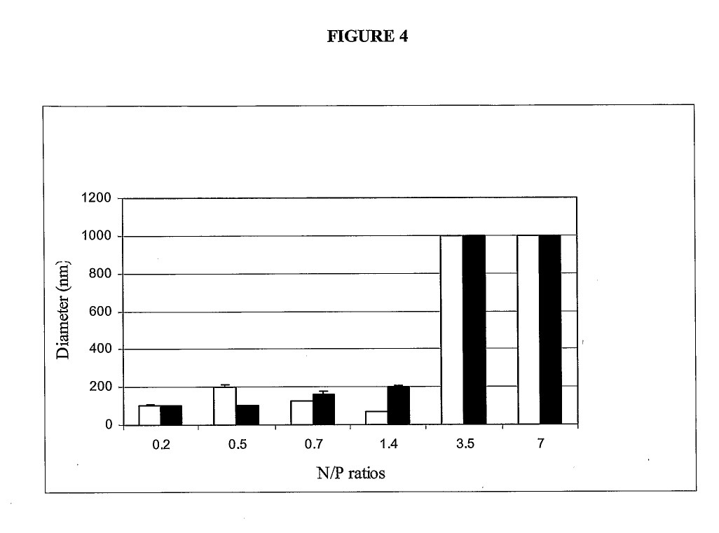

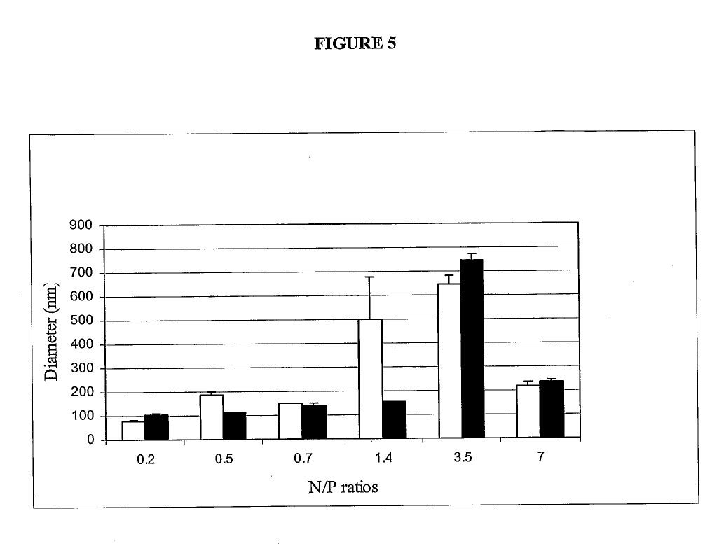

Diameters of particles of condensate compounds of siRNA G1498 and peptide PN183 obtained at various times after mixing and at various nitrogen to phosphorous ratios (N/P) are shown in FIGS. 2-5. In each of FIGS. 2-5, for each group of two bars at a particular N/P ratio, the left bar was with vortexing, while the right bar was without vortexing.

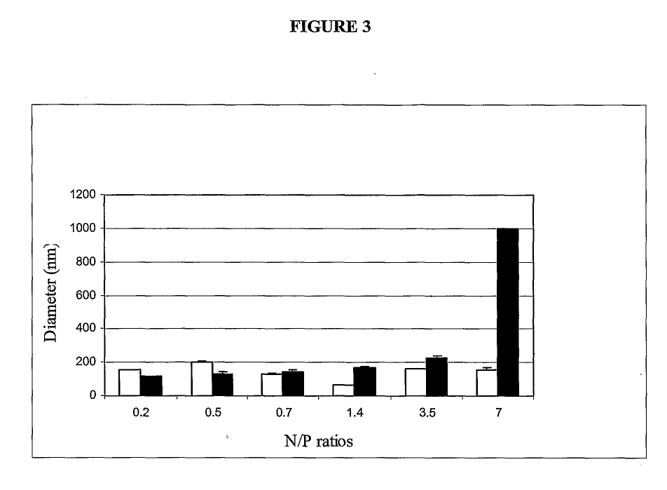

The particles sizes in FIG. 2 were obtained immediately after mixing, while those of FIGS. 3, 4, and 5 were obtained 30 minutes, 60 minutes, and 24 hours after mixing, respectively.

### Example 4

### Effect of pH on Condensate Particle Size

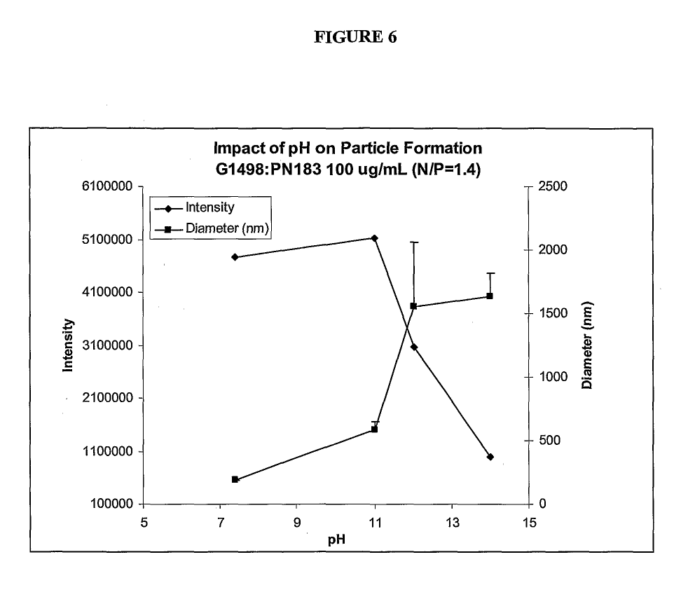

Diameters of particles of condensate compounds of siRNA G1498 and peptide PN183 obtained at an N/P ratio of 1.4 and at a concentration of G1498 of 100 ug/ml for various values of pH are shown in FIG. 6.

At pH below about 12, condensate particle size decreases, and continues to decrease as pH decreases. Particle size was below about 500 nm for pH below about 11.

The intensity is the measure of back scattered photons (backscatter mode). The particle size is calculated size using an algorithm for the diffusion autocorrelation.

### Example 5

### Effect of Salt Concentration on Condensate Particle Size

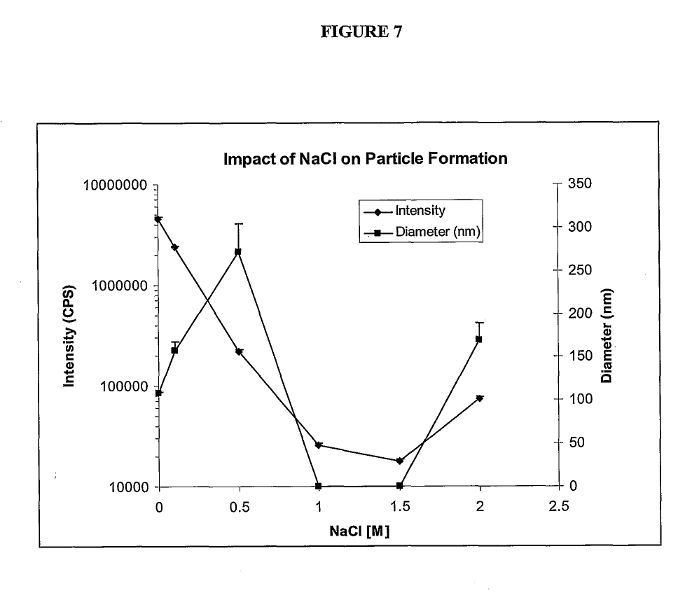

Diameters of particles of condensate compounds of siRNA G1498 and peptide PN183 obtained at various concentrations of sodium chloride are shown in FIG. 7.

At sodium chloride concentrations up to about 0.5, particle size increases from about 100 nm to about 275 nm. At sodium chloride concentrations greater than about 0.5, condensate particle size fluctuates.

### Example 6

### Effect of Order of Addition of RNA and Peptide on Condensate Particle Size

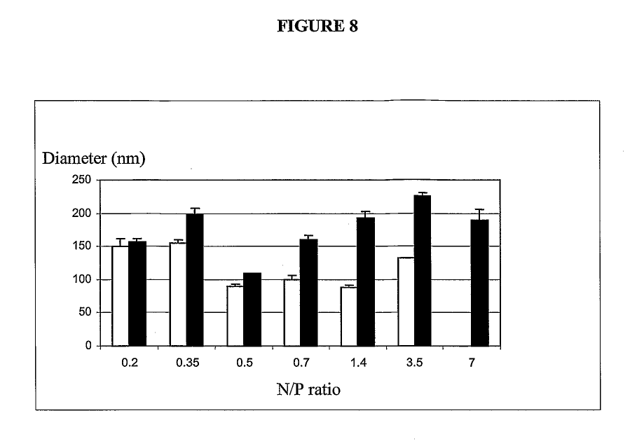

Diameters of particles of condensate compounds of siRNA G1498 with peptide PN183 at various N/P ratios and order of mixing are shown in FIG. 8. At N/P ratios at or below about 0.5, particle size was not much affected by the order of addition. At N/P ratios above about 0.5, particle size was generally smaller when the siRNA was introduced to the solution first, and the peptide was added to the siRNA solution.

### Example 7

### Morphology of Condensate Particles

The morphology of the particles of a peptide-RNA condensate compound was determined by transmission electron microscopy (TEM) imaging. The following protocol was used:

15 uL sample drop on top of grid, 10 min;

Half strength Karnofsky's solution dip;

Cacodylate buffer dip;

TEM Contrast agent: 3% Uranyl Acetate (UA);

Water dip, 3×, UA dip, blot off excess on damp filter paper, dry.

Mixture 1: (Original Karnovsky's Mixture);

16% Paraformaldehyde Solution: 20 mL;

50% Glutaraldehyde EM Grade: 8 mL;

0.2M Sodium Phosphate Buffer: 25 mL;

Distilled Water: 25 mL.

Final mixture is 78 mL with 5% Glutaraldehyde, 4% Formaldehyde in 0.08M buffer.

This mixture had an osmolarity of more than 2000 m OSM.

Sodium Cacodylate Buffers 0.1M;

Sodium cacodylate: 4.28 gm;

Calcium chloride: 25.0 gm;

0.2N hydrochloric acid: 2.5 ml;

Dilute to 200 ml with distilled water, pH 7.4.

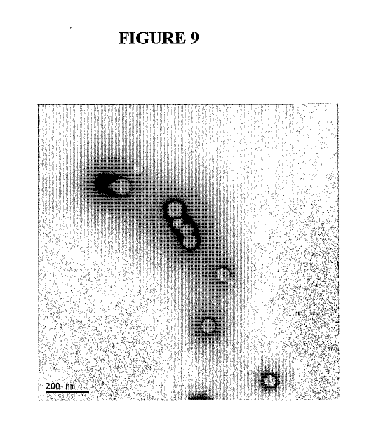

Using the protocol above with no glow discharge, a TEM of particles of a condensate compound of siRNA G1498 (concentration 100 ug/ml) with peptide PN183 (N/P=1.4) is shown in FIG. 9. This image shows particles of uniform size and spherical morphology. The particle size was below 100 nm, typically about 50-60 nm.

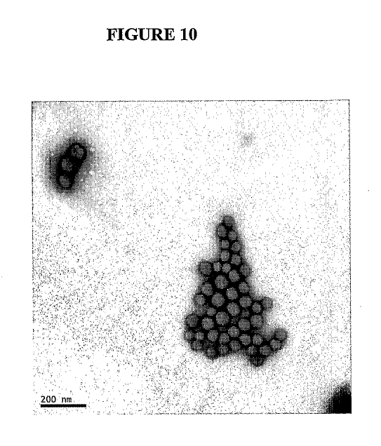

Using the protocol above with glow discharge, a TEM of particles of a condensate compound of siRNA G1498 (concentration 100 ug/ml) with peptide PN183 (N/P=1.4) is shown in FIG. 10. This image shows particles of uniform size and spherical morphology. The particle size is below 100 nm, typically about 30-60 nm.

### Example 8

### Particle Size Characteristics of Peptide-RNA Condensate Compounds

The particle size characteristics of some peptide-RNA condensate compounds are summarized in Table 5.

TABLE 5
Particle Size Characteristics of Peptide-RNA Condensates

| | | Particle sizes at half | | Z-avg. | | Zeta |
|---|---|---|---|---|---|---|
| | | peak height in nm (% | | diameter | | Potenial |
| Compound | N/P | of population) | | (nm) | | (mV) |
| G1498/PN183 | 0.5 | 40-106 | (98.7%) | 63.8 | | −35.7 |
| | | | | | | (100%) |
| G1498/PN183 | 2 | 50-110 | (100%) | 93.2 | | 27.9 |
| | | | | | | (100%) |
| G1498/PN826 | 2 | 103-120 | (92.7%) | 189 | peak | 34.6 |
| G1498/PN183/ | 0.5; 1 | 90-145 | (47.4%) | 119 | peak | 31.3 |
| PN826 | | 190-340 | (52.6%) | 268 | peak | (100%) |
| G1498/PN861 | 2 | 180-330 | (98.7%) | 241 | | — |
| G1498/PN939 | 2 | 20-70 | (92.1%) | 37.0 | | — |
| G1498/PN938 | 2 | <10 | (32.3%) | <10 | | — |
| | | 180-500 | (56.8%) | 283 | peak | |
| G1498/PN183/ | 0.5; 1 | <1 | (9.8%) | 0.8 | peak | — |
| PN939 | | 15-35 | (68.5%) | 23.2 | peak | |
| | | 200-400 | (21.7%) | 274 | peak | |
| G1498/PN924 | 2 | <1 | (41.4%) | 0.8 | peak | — |
| | | 5-8 | (11.3%) | 6.2 | peak | |
| | | 80-200 | (47.3%) | 135 | peak | |

| | | | | | |
|---|---|---|---|---|---|
| G1498/PN859 | 2 | 530-770 | (94.7%) | 702 | — |

For example, compound G1498/PN183 at N/P of 0.5 exhibited a peak having 98.7% of total intensity, 73.3 nm peak diameter, 32.9 mm peak width, and Z-average diameter 63.8.

### Example 9

### In Vivo Knockdown Assay of TFN-α in LPS-Stimulated Mouse Lung Using Peptide-RNA Condensates

SiRNA knockdown activity was determined by transfecting cells with a peptide-siRNA condensate compound. A random siRNA sequence was used as a negative control.

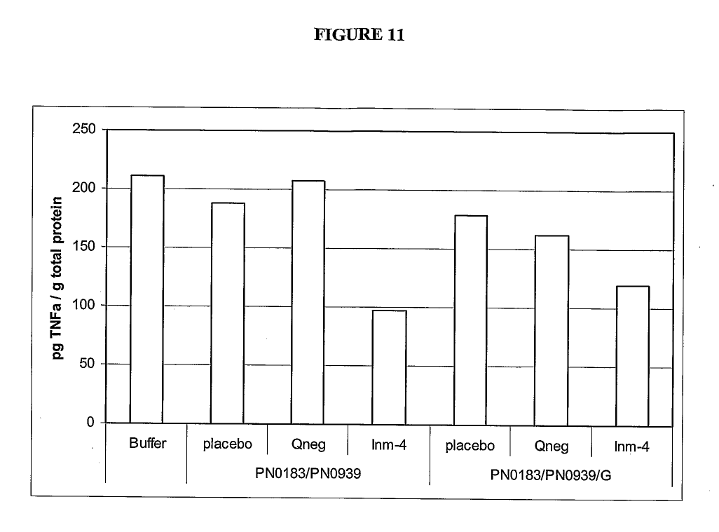

FIG. 11 shows the results of a knockdown assay of LPS-induced TFN-α expression (pg/ml) in a mouse model by intranasal administration of a composition including a condensate compound of siRNA Inm-4 and peptides PN183 and PN939.

In FIG. 11, buffer control is the leftmost bar, followed by data for condensate Inm-4/PN183/PN939, and followed on the right by data for compound Inm-4/PN183/PN939 crosslinked with glutaraldehyde (G). Placebo does not contain the siRNA, and Qneg contains a non-active-siRNA.

The data for FIG. 11 are given in Table 6.

TABLE 6
Knockdown of TFN-α in LPS-Stimulated Mouse Lung With Inm-4 siRNA

| | | LPS Lung Assay | |
|---|---|---|---|
| Compound | Assay | Mean | SD |
| Buffer (10 mM Hepes/5% Dextrose) | | 211.3 | 25 |
| PN183/PN939 | Placebo | 188.3 | 10 |
| N/P = 0.75/0.5 | Qneg | 207.1 | 95 |
| | Inm-4 agent | 97.0 | 55 |
| PN183/PN939/G | Placebo | 179.1 | 51 |
| N/P = 0.75/0.5 | Qneg | 161.7 | 108 |
| G = 0.8 ME | Inn-4 agent | 119.4 | 6 |

Dose was administered intranasally. At 4 and 24 hrs post dose, animals were induced with 0.625 ng LPS (50 μl). Lungs were collected 2 hrs. post-LPS. A TNFα ELISA assay and a BCA total protein assay were performed. Materials and methods for the assay were as follows:
Animals: Normal mice
Dose: 0.5 mg/kg
Volume: 50 uL
Replicates: n=3
Total Groups: 10
Controls: Vehicles, Qneg
siRNA: Inm4
Dosing: Formulations were prepared at 0.5 mg/kg with Inm4. Each formulation had 200 ul in total. Each mouse (n=3) received 50 ul.
siRNA preparation: Existing 20 mg/ml stock of Inm-4 siRNA was diluted into 5 mg/ml using Hepes/Buffer. Existing 3.29 mg/ml stock of Qneg was used.
Peptides: Peptides were diluted to appropriate concentration using Buffer (10 mM Hepes/5% dextrose).
Excipient Preparation Glutaraldehyde (0.05% W/V) was used, 0.2 um filtered to be sterile.
Formulation Preparation: Components were added to Bio-pur Eppendorf 1.5 ml tubes.
(A) Buffer was added first, as a receiving volume for small volumes of other components.
(B) All the components were added in the following order: siRNA, peptide-1, peptide-2, additive, if any, buffer.
(C) For formulations with glutaraldehyde crosslinking, waited for one hour before dosing.

Representative formulations are given in Table 7.

TABLE 7
Formulations for Peptide-siRNA Compounds

| | Vol. of | Vol. of | Vol. of | | | |
|---|---|---|---|---|---|---|
| | siRNA | peptide | peptide | Vol. of | Vol. of | Total |
| | stock | stock-1 | stock-2 | additive | Buffer | Vol. |
| Formulation | (ul) | (ul) | (ul) | (ul) | (ul) | (ul) |
| Buffer (10 mM Hepes, 5% | 0 | 0 | 0 | 0 | 200 | 200 |
| Dextrose) | | | | | | |
| PN0183/PN0939 | 0.00 | 11.20 | 6.80 | 0.00 | 182.00 | 200 |
| N/P = 0.75, N/P = 0.5 | | | | | | |
| PN0183/PN0939/G | 0.00 | 11.20 | 6.80 | 34.48 | 147.52 | 200 |
| N/P = 0.75, N/P = 0.5, 0.8 | | | | | | |
| mole equivalent | | | | | | |
| Q.Neg/PN0183/PN0939 | 15.20 | 11.20 | 6.80 | 0.00 | 166.80 | 200 |
| N/P = 0.75, N/P = 0.5 | | | | | | |
| Q.Neg,/PN0183/PN0939/G | 15.20 | 11.20 | 6.80 | 34.48 | 132.32 | 200 |
| N/P = 0.75, N/P = 0.5, 0.8 | | | | | | |
| mole equivalent | | | | | | |
| Inm4/PN0183/PN0939 | 10.00 | 11.20 | 6.80 | 0.00 | 172.00 | 200 |
| N/P = 0.75, N/P = 0.5 | | | | | | |
| Inm4/PN0183/PN0939/G | 10.00 | 11.20 | 6.80 | 34.48 | 137.52 | 200 |
| N/P = 0.75, N/P = 0.5, 0.8 | | | | | | |
| mole equivalent | | | | | | |

Details of the representative formulations are given in Tables 8 and 9.

TABLE 8

| | |
|---|---|
| siRNA final concentration = | 250 ug/ml (0.5 mg/kg) |
| siRNA Inm4 stock conc. = | 5 mg/ml |
| Qneg. = | 3.29 mg/ml |
| PN0183 = | 5 mg/ml |
| PN0939 = | 5 mg/ml |
| Glutaraldehyde = | 0.05% W/V |

TABLE 9

| Materials | Batch ID | Concentration |
|---|---|---|
| Inm4 | BS31 | 20 mg/ml |
| Q.Neg | B324P167 | 3.29 mg/ml |
| PN0183-2 | BP1 | 10 mg/ml |
| PN0939-2 | BP9 | 20 mg/ml |
| Glutaraldehyde | BR39 | 0.05% W/V |
| Buffer | BB72 | 10 mM Hepes/5% Dextrose |

### Example 10

### In Vitro Knockdown Assay of Lac-z Expression in Rat Gliosarcoma Fibroblast Cells (9 L/LacZ)

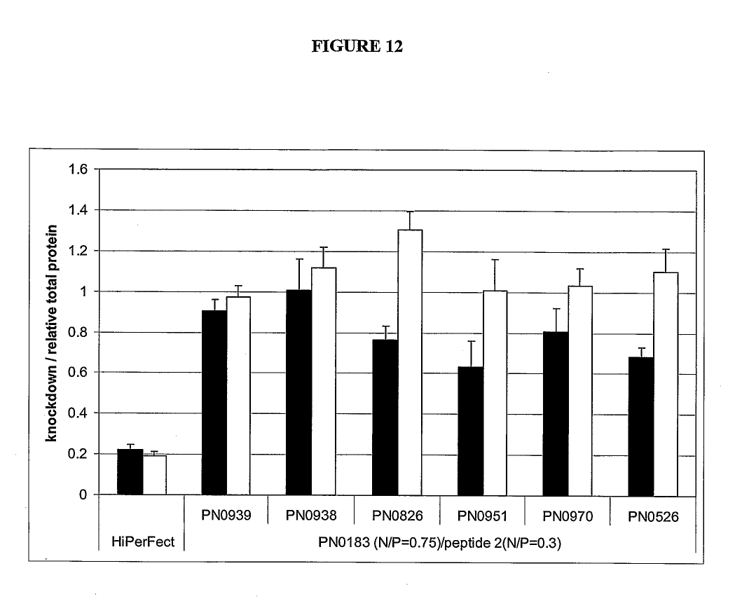

FIG. 12 shows the results of a knockdown in vitro assay of lac-z expression in rat gliosarcoma fibroblast cells 9 L/LacZ for condensate compounds of the lac-z siRNA with peptide PN183 and various second peptides.

In FIG. 12, comparative data using HiPerFect™ (Qiagen; Valencia, Calif.) is the leftmost bar, followed by data for various compounds of this invention. The N/P ratio for PN183 was 0.75, while the N/P ratio for the second peptide was 0.3. The data for FIG. 12 are given in Table 10.

TABLE 10
Lac-z Knockdown in vitro Assay

| | | Knockdown | |
|---|---|---|---|
| | | mean | SD |
| HiPerFect | | 0.221048 | 0.028369 |
| PN0183 (N/P = 0.75)/ | PN0939 | 0.905998 | 0.053035 |
| peptide 2 (N/P = 0.3) | PN0938 | 1.007354 | 0.1546 |
| | PN0826 | 0.762651 | 0.069725 |
| | PN0951 | 0.629382 | 0.128045 |
| | PN0970 | 0.806908 | 0.11293 |
| | PN0526 | 0.682695 | 0.045614 |

Materials and methods for the assay were as follows:
Cells: 9 L/LacZ
Dose: 100 nM; based on 100 ul total transfection volume.
Volume: 25 uL formulation volume.
Replicates: n=3.
Total Groups: 20.
Controls: Qneg w/Alexis 546.
siRNA: LacZ.
Lac-Z or Qneg: 54 ul siRNA+17.28 ul PN0183+1278 ul OPTI-MEM.
Peptides were diluted into appropriate concentration using OPTI-MEM medium. All excipients were 0.2 um filtered to be sterile.

Formulation:
(A) Diluted siRNA and PN0183 together using OPTI-MEM to form particles. Vortexed.
(B) Diluted delivery vehicles using OPTI-MEM. Vortexed to mix the delivery vehicle.
(C) For each formulation, in 96 well, added diluted delivery vehicle first and then added siRNA/PN0183 formulation. Pipetted to mix. Waited for 30 mins. before sending for transfection. Transfection: Each formulation was 125 ul which was enough for 5 wells. Each well (n=3) received 25 ul.

Representative formulations are given in Table 11.

TABLE 11

| | | | | | Total vol |
|---|---|---|---|---|---|
| | | | | Vol of | for |
| | Vol of | Vol of | | Optimem | delivery |
| | peptide | peptide | Vol of | for addtive | substances |
| | stock1 for | stock2 for | additives | dilution | dilution |
| Formul. | 6 wells | 10 wells | for 10 | (10 well; | (10 well; |
| with PN183 | (ul) | (ul) | wells (ul) | ul) | ul) |
| PN939 | 0.96 | 5.95 | 0.00 | 119.05 | 125.00 |
| PN938 | 0.96 | 2.91 | 0.00 | 122.09 | 125.00 |
| PN826 | 0.96 | 3.96 | 0.00 | 121.04 | 125.00 |
| PN951 | 0.96 | 3.12 | 0.00 | 121.88 | 125.00 |
| PN970 | 0.96 | 25.85 | 0.00 | 99.15 | 125.00 |
| PN526 | 0.96 | 6.90 | 0.00 | 118.10 | 125.00 |

Details of the representative formulations are given in Table 12.

TABLE 12

| Materials | Batch ID | Concentration | |
|---|---|---|---|
| Lac-Z | Qiagen (Cat 1027020; Lot 161545/161546); 20 uM | | |
| Q.Neg | Qiagen (Cat 1027098; Lot 160427/160428); 20 uM | | |
| (Alexis 546) | | | |
| PN0183-2 | BP86 | 7.26 | mg/ml |
| PN0939-2 | BP9 | 20 | mg/ml |
| PN0826-2 | BP2 | 5 | mg/ml |
| PN0951-2 | BP10 | 10 | mg/ml |
| PN0970-1 | 462-124 | 2 | mg/ml |
| PN0526-2 | | 3.91 | mg/ml |

| | | |
|---|---|---|
| Buffer | BB72 | 10 mMHepes/5% Dextrose |

### Example 11

### In Vitro Knockdown Assay of Lac-z Expression in Rat Gliosarcoma Fibroblast Cells (9 L/LacZ)

Table 13 shows the results of knockdown in vitro assays of lac-z expression in rat gliosarcoma fibroblast cells 9 L/LacZ for various condensate compounds.

TABLE 13
Knockdown of Lac-z Expression in Rat Model Cell Line 9L/LacZ

| | | | | Relative | |
|---|---|---|---|---|---|
| | | | | protein | |
| | | | | concentration | |
| | N/P | LacZ assay | | (Qneg) | |
| Compound | ratios | Mean | SD | Mean | SD |
| HiPerFect ™ | — | 0.165 | 0.028 | 0.413 | 0.057 |
| PN0183/G (0.25 mol.eq., | 0.75/2 | 0.510 | 0.071 | 1.059 | 0.121 |
| dialyzed)/PN0951 | | | | | |
| PN0183/G (0.25 mol.eq., | 0.75/5 | 0.613 | 0.194 | 1.051 | 0.150 |
| dialyzed)/PN0951 | | | | | |
| PN0183/G (0.25 mol.eq., | 0.75/0.5 | 0.725 | 0.129 | 1.146 | 0.183 |
| dialyzed)/PN0939 | | | | | |
| PN0183/G (5 mol.eq., | 0.75/0.5 | 0.524 | 0.107 | 1.218 | 0.042 |
| dialyzed)/PN0939 | | | | | |

Materials and methods for the assay were as follows:
Cells: 9 L/LacZ.
Dose: 100 nM; based on 100 ul total transfection volume.
Volume: 25 uL formulation volume.
Replicates: n=3.
Total Groups: 20.
Controls: Qneg w/Alexis 546.
siRNA: LacZ.
Transfection: Each formulation had 125 ul which was enough for 5 wells. Each well (n=3) received 25 ul.
Peptide Preparation Peptide was diluted into appropriate concentration using OPTI-MEM medium.
Excipient Preparation All the excipients were 0.2 um filtered to be sterile.

Formulation Preparation:
(A) For formulations without PN0183, added delivery vehicle first and then siRNA, pipetted to mix.
(B) For formulations with PN0183, made siRNA and PN0183 complex first. In 96 well plate, added delivery vehicles first, and then siRNA/PN0183 complex, pipetted to mix.
(C) For formulations with crosslinking, make the siRNA/PN0183 complex first, then either dialysis (at 4°, overnight), or without dialysis. Then next morning, added other delivery vehicles first and siRNA/PN0183 complex, and then pipetted to mix.

Representative formulations are given in Table 14.

TABLE 14

| | | | Vol of | |
|---|---|---|---|---|
| | | Volume of | Optimem for | Total vol for |
| | | delivery | delivery | delivery |
| | | reagents for | reagents | substances |
| | | 10 wells | dilution (10 | dilution (10 |
| Code | Formulation | (ul) | well; ul) | well; ul) |
| U | PN0183/G/PN0951 | 20.82 | 104.18 | 125.00 |
| | N/P = 0.75, 0.25 ME, | | | |
| | D, N/P = 2 | | | |
| V | PN0183/G/PN0951 | 52.05 | 72.95 | 125.00 |
| | N/P = 0.75, 0.25 ME, | | | |
| | D, N/P = 5 | | | |
| W | PN0183/G/PN0939 | 9.92 | 115.08 | 125.00 |
| | N/P = 0.75, 0.25 ME, | | | |
| | D, N/P = 0.5 | | | |
| Y | PN0183/G/PN0939 | 9.92 | 115.08 | 125.00 |
| | N/P = 0.75, 5 ME, | | | |
| | D, N/P = 0.5 | | | |

Details of the representative formulations are given in Tables 15, 16, and 17.

TABLE 15

Crosslinking: Prepared siRNA/PN0183 complex first.
0.25 ME crosslinking: 103.45 ul of stock siRNA (20 uM) + 33.53 ul
PN0183 (1 mg/ml) + 4.4 Glutar. (0505%) + 158.6 ul Hepes Buffer.
5 ME crosslinking: 68.97 ul siRNA stock (20 uM) + 22.35 ul PN0183
(1 mg/ml) + 5.9 ul of Glutar. (0.5%) + 102.78 ul Hepes Buffer.
5 ME without dialysis: 17.24 ul siRNA (20 uM) + 5.59 ul PN0183
(1 mg/ml) + 1.48 ul of Glutar. (0.5%) + 25.69 ul Hepes Buffer.
For 0.25 ME crosslinking: 43.5 ul crosslinked complex + 331.5
OPTI-MEM.
For 5 ME crosslinking: 8.7 ul crosslinked complex + 66.3 ul
OPTI-MEM.

TABLE 16

| | |
|---|---|
| G = | Glutaraldehyde |
| ME = | Molar Equivalent |
| D = | Dialysis |
| Crosslinking = | 2 hrs at room temperature |
| PN0951 = | 0.1 mg/ml |
| PN0939 = | 0.1 mg/ml |
| OPTI-MEM | |

TABLE 17

| Materials | Batch ID | Concentration | |
|---|---|---|---|
| Lac-Z | Qiagen (Cat 1027020; Lot 161545/161546); 20 uM | | |
| Q.Neg | Qiagen (Cat 1027098; Lot 160427/160428); 20 uM | | |
| (Alexis 546) | | | |
| PN0951-2 | BP10 | 10 | mg/ml |
| PN0183-2 | BP86 | 7.26 | mg/ml |
| PN0939 | BP9 | 20 | mg/ml |

| Buffer | | 10 mMHepes/5% Dextrose, pH 7.4 |
|---|---|---|
| OPTI-MEM | BB57 | |

### Example 12

### Polynucleotide Delivery-Enhancing Polypeptides

The exemplary polynucleotide delivery-enhancing polypeptide PN73 was derived from the amino acid sequence of the human histone 2B (H2B) protein, which is shown below. The underlined residues 13 through 48 found within H2B protein identify the fragment used to derive PN73. It may also be represented by H2B amino acids 12 through 48. The primary structure of PN73 is also shown below.

H2B(histone 2B) amino acid sequence
(SEQ ID NO: 66)
MPEPAKSAPAPKKGSKKAVTKAQKKDSKKRKRSRKESYSVYVYKVLKVH
PDTGISSKAMGIMNSFVNDIFERIAGEASRLAHYNKRSTITSREIQTAV
RLLLPGELAKHAVSEGTKAVTKYTSSK
PN73 (13-48)
(SEQ ID NO: 42)
NH2-KGSKKAVTKAQKKDGKKRKRSRKESYSVYVYKVLKQ-amide

Table 18 shows the structure of some mutant polynucleotide delivery-enhancing polypeptides made by residue substitutions and deletions of the exemplary polynucleotide delivery-enhancing polypeptide PN73.

TABLE 18
PN73 Residue Substitution and Deletion Series

| | SEQ | |
|---|---|---|
| | ID | |
| Peptide | NO: | Amino Acid Sequence |
| PN73 | 42 | KGSKKAVTKAQKKDGKKRKRSRKESYSVYVYKVLKQ |
| PN644 | 67 | KGSKKAVTKAQKKDGKKRKRSRKESYWVYVYKVLKQ |
| PN645 | 68 | KGSKKAVTKAQKKDGKKRKRSRKWSYSVYVYKVLKQ |
| PN646 | 69 | KGSKKAVTKAQKKDGKKRKRSRKFSYSVYVYKVLKQ |
| PN647 | 70 | KGSFKAVTKAQKKDGKKRKRSFKFSYSVYVYKVLKQ |
| PN729 | 71 | KGSFKAVTKAQKKFGKKRKRSRKSFSVYVYKVLKQ |

Table 19 shows the structure of exemplary polynucleotide delivery-enhancing polypeptide PN73 and truncated derivatives thereof. The amino acids sequence for PN360 and PN361 listed below are aligned with the corresponding amino acid sequence of PN73.

TABLE 19
PN73 Deletion Series

| | | SEQ | |
|---|---|---|---|
| C-Term. | | ID | |
| Label | Peptide | NO: | Amino Acid Sequence |
| None | PN73 | 42 | KGSKKAVTKAQKKDGKKRKRSRKESYSVYVYKVLKQ-amide |
| | PN360 | 72 | KGSKKAVTKAQKKDGKKRKRSRK-amide |
| | PN361 | 73 | KKDGKKRKRSRKESYSVYVYKVLKQ -amide |
| | PN766 | 74 | RKESYSVYVYKVLKQ-amide |
| | (PN708) | | |
| FTTC | PN690 | 75 | KGSKKAVTKAQKKDGKKRKRSRKESYSVYVYKVLKQ-GK[EPSILON-5CFG- |
| (fluorescein- | (PN73) | | amide |
| 5- | PN661 | 76 | KKAVTKAQKKDGKKRKRSRKESYSVYVYKVLKQ-GK[EPSILON-5CFG-amide |
| isothiocyanate) | PN685 | 77 | VTKAQKKDGKKRKRSRKESYSVYVYKVLKQ-GK[EPSILON-5CFG amide |
| label | PN660 | 78 | AQKKDGKKRKRSRKESYSVYVYKVLKQ-GK[EPSILON-5CFG-amide |
| (i.e.,- | PN735 | 79 | KDGKKRKRSRKESYSVYVYKVLKQ-GK[EPSILON-5CFG-amide |
| GK[EPSILON]G- | PN655 | 80 | KKRKRSRKESYSVYVYKVLKQ-GK[EPSILON-5CFG-amide |
| amide) | PN654 | 81 | KRSRKESYSVYVYKVLKQ-GK[EPSILON-5CFG-amide |
| | PN708 | 82 | RKESYSVYVYKVLKQ-GK[EPSILON-5CFG-amide |
| | PN653 | 83 | SYSVYVYKVLKQ-GK[EPSILON-5CFG-amide |
| | PN652 | 84 | VYVYKVLKQ-GK[EPSILON-5CFG-amide |
| | PN651 | 85 | YKVLKQ-GK[EPSILON-5CFG-amide |
| | PN768 | 86 | KVLKQ-GK[EPSILON-5CFG-amide |

PN360 shares its N-terminus with PN73 but lacks PN73's C-terminus while PN361 shares its C-terminus with PN73 but lacks PN73's N-terminus. PN766 represents the 15 C-terminal amino acids of PN73. PN73, PN360, PN361 and PN766 are not tagged with a C-terminal FITC (fluorescein-5-isothiocyanate) (i.e., -GK[EPSILON]G-amide). Table 19 further shows the 11 truncated forms of PN73 that were created by sequentially deleting 3 residues at a time, except PN768, from the N-terminus of the peptide. All these peptides were tagged with a C-terminus FITC (fluorescein-5-isothiocyanate) label (i.e., -GK[EPSILON]G-amide) so that cells containing the peptide could be detected by fluorescent microscopy and/or sorted by flow cytometry. PN766 and PN708 have the same amino acid sequence but differ in that PN708 has the C-terminus FITC tag.

### Example 13

### In Vitro Methods and Procedures for siRNA Cell-Uptake and Target Gene Knockdown

The present example illustrates the methods and procedures used to assess the efficacy of the exemplary polynucleotide delivery-enhancing polypeptides listed in Table 18 and Table 19 of Example 12 to enhance siRNA cell-uptake and siRNA mediated target gene knockdown activities. Cell viability was also assessed. The cell culture conditions and protocols for each assay are explained below in detail.

### Cell Cultures

Primary Human Monocytes: Fresh human blood samples from healthy donors were purchased from Golden West Biologicals. For isolation of monocytes, blood samples were diluted with PBS at a 1:1 ratio immediately after receiving. Peripheral blood mononuclear cells (PBMC) were first isolated by Ficoll (Amersham) gradient from whole blood. Monocytes were further purified from PBMCs using the Miltenyi CD14 positive selection kit and supplied protocol (MILTENYI BIOTEC). To assess the purity of the monocyte preparation, cells were incubated with an anti-CD14 antibody (BD Biosciences) and then sorted by flow cytometry. The purity of the monocyte preparation was greater than 95%.

Activation of human monocytes was performed by adding 0.1-1.0 ng/ml of Liposaccharides, LPS (Sigma, St Louis, Mo.) to the cell culture to stimulate tumor necrosis factor-± (TNF-±) production. Cells were harvested 3 hours after incubation with LPS and mRNA levels were determined by Quantigene assay (Genospectra, Fremont, Calif.) according to the manufacturer's instructions.

Mouse Tail Fibroblast Cells: Mouse tail fibroblast (MTF) cells were derived from the tails of C57BL/6J mice. Tails were removed, immersed in 70% ethanol and then cut into small sections with a razor blade. The sections were washed three times with PBS and then incubated in a shaker at 37° C. with 0.5 mg/mL collagenase, 100 units/mL penicillin and 100 μg/mL streptomycin to disrupt tissue. Tail sections were then cultured in complete media (Dulbecco's Modified Essential Medium with 20% FBS, 1 mM sodium pyruvate, nonessential amino acids and 100 units/mL penicillin and 100 μg/mL streptomycin) until cells were established. Cells were cultured at 37° C., 5% CO₂ in complete media as outlined above.

### Cell Viability (MTT Assay)

Cell viability was assessed using the MTT assay (MTT-100, MatTek kit). This kit measures the uptake and transformation of tetrazolium salt to formazan dye. Thawed and diluted MTT concentrate was prepared 1 hour prior to the end of the dosing period with the lipid by mixing 2 mL of MTT concentrate with 8 mL of MTT diluent. Each cell culture insert was washed twice with PBS containing Ca⁺² and Mg⁺² and then transferred to a new 96-well transport plate containing 100 μL of the mixed MTT solution per well. This 96-well transport plate was then incubated for 3 hours at 37° C. and 5% CO₂. After the 3 hour incubation, the MTT solution was removed and the cultures transferred to a second 96-well feeder tray containing 250 μL MTT extractant solution per well. An additional 150 μL of MTT extractant solution was added to the surface of each culture well and the samples sat at room temperature in the dark for a minimum of 2 hours and maximum of 24 hours. The insert membrane was then pierced with a pipet tip and the solutions in the upper and lower wells were allowed to mix. Two hundred microliters of the mixed extracted solution along with extracted blanks (negative control) was transferred to a 96-well plate for measurement with a microplate reader. The optical density (OD) of the samples was measured at 570 nm with the background subtraction at 650 nm on a plate reader. Cell viability was expressed as a percentage and calculated by dividing the OD readings for treated inserts by the OD readings for the PBS treated inserts and multiplying by 100. For the purposes of this assay, it was assumed that PBS had no effect on cell viability and therefore represented 100% cell viability.

siRNA Preparation

Synthesis of oligonucleotides was carried out using the standard 2-cyanoethyl phosphoramidite method (1) on long chain alkylamine controlled pore glass derivatized with 5′-O-Dimethyltrityl-2′-O-t-butyldimethylsilyl-3′-O-succinyl ribonucleoside of choice or 5′-O-Dimethyltrityl-2′-deoxy-3′-O-succinyl thymidine support where applicable. All oligonucleotides were synthesized at either the 0.2 or 1-μmol scale using an ABI 3400 DNA/RNA synthesizer (Applied Biosystems, Foster City, Calif.), cleaved from the solid support using concentrated NH₄OH, and deprotected using a 3:1 mixture of NH₄OH:ethanol at 55° C. The deprotection of 2′-TBDMS protecting groups was achieved by incubating the base-deprotected RNA with a solution (600 μL per μmol) of N-methylpyrrolidinone/triethylamine/triethylamine trihydrofluoride (NMP/TEA/3HF; 6:3:4 by volume) at 65° C. for 2.5 hours. The corresponding building blocks, 5′-dimethoxytrityl-N-(tac)-2′-O-(t-butyldimethylsilyl)-3′-[(2-cyanoethyl)-(N,N-diisopropyl)]-phosphoramidites of A, U, C and G (Proligo, Boulder, Colo.) as well the modified phosphoramidites, 5′-DMTr-5-methyl-U-TOM-CE-Phosphoramidite, 5′-DMTr-2′-OMe-Ac—C-CE Phosphoramidite, 5′-DMTr-2′-OMe-G-CE Phosphoramidite, 5′-DMTr-2′-OMe-U-CE Phosphoramidite, 5′-DMTr-2′-OMe-A-CE Phosphoramidite (Glen Research, Sterling Va.) were purchased directly from suppliers. Triethylamine-trihydrofluride, N-methylpyrrolidinone and concentrated ammonium hydroxide was purchased from Aldrich (Milwaukee, Wis.). All HPLC analysis and purifications were performed on a Waters 2690 HPLC system with Xterra™ C18 columns. All other reagents were purchased from Glen Research Inc. Oligonucleotides were purified to greater than 97% purity as determined by RP-HPLC. siRNAs for mouse injection were purchased from Qiagen (Valencia, Calif.) as in-vivo grade, which were HPLC purified after annealing. The amount of single stranded siRNA was determined spectrophotometrically based on a calculated extinction coefficient of 35.0 μg/OD for the sodium salt form at λ=260 nm. When the two strands are annealed, approximately 10% hypochromicity was observed; therefore the extinction coefficient was lowered by 10% for the quantitation of the double stranded forms. Endotoxin levels of siRNAs were typically equal to or less than 0.0024 EU/mg.

### Peptide Synthesis

Peptides were synthesized by solid-phase Fmoc chemistry on CLEAR-amide resin using a Rainin Symphony synthesizer. Coupling steps were performed using 5 equivalents of HCTU and Fmoc amino acid with an excess of N-methylmorpholine for 40 minutes. Fmoc removal was accomplished by treating the peptide resin with 20% piperidine in DMF for two 10 minutes cycles. Upon completion of the entire peptide, the Fmoc group was removed with piperidine and washed extensively with DMF. Maleimido modified peptides were prepared by coupling 3.0 equivalents of 3-maleimidopropionic acid and HCTU in the presence of 6 equivalents of N-methylmorpholine to the N-terminus of the peptide resin. The extent of coupling was monitored by the Kaiser test. The peptides were cleaved from the resin by the addition of 10 mL of TFA containing 2.5% water and 2.5 triisopropyl silane followed by gentle agitation at room temperature for 2 h. The resulting crude peptide was collected by trituration with ether followed by filtration. The crude product was dissolved in Millipore water and lyophilized to dryness. The crude peptide was taken up in 15 mL of water containing 0.05% TFA and 3 mL acetic acid and loaded onto a Zorbax RX-C8 reversed-phase (22 mm ID×250 mm, 5 μm particle size) through a 5 mL injection loop at a flow rate of 5 mL/min. The purification was accomplished by running a linear AB gradient of 0.1% B/min where solvent A is 0.05% TFA in water and solvent B is 0.05% TFA in acetonitrile. The purified peptides were analyzed by HPLC and ESMS.

### Flow Cytometry

Fluorescence activated cell sorting (FACS) analysis were performed using Beckman Coulter FC500 cell analyzer (Fullerton, Calif.). The instrument was adjusted according to the fluorescence probes used (FAM or Cy5 for siRNA and FITC and PE for CD14). Propidium iodide (Fluka, St Louis) and AnnexinV (R&D systems, Minneapolis) were used as indicators for cell viability and cytotoxicity. A brief step-by-step protocol is detailed below.
(a) After exposure to the complex of siRNA/peptide, cells were incubated for at least 3 hours.
(b) Wash cells with 200 μl PBS.
(c) Detach cells with 15 μl TE, incubate at 370 C.
(d) Re-suspend cells in five wells with 30 μl FACS solution (PBS with 0.5% BSA, and 0.1% sodium Azide).
(e) Combine all five wells into a tube.
(f) Add PI (Propidium iodide) 5 μl into each tube.
(g) Analyze the cells with fluorescence activated cell sorting (FCAS) according to manufacturer's instructions.

For siRNA uptake analysis, cells were washed with PBS, treated with trypsin (attached cells only), and then analyzed by flow cytometry. Uptake of the siRNA designated BA, described above, was also measured by intensity of Cy5 or FITC fluorescence in the cells and cellular viability assessed by addition of propidium iodide or AnnexinV-PE. In order to differentiate the cellular uptake from the membrane insertion of fluorescence labeled siRNA, trypan blue was used to quench the fluorescence on the cell membrane surface.

### Example 14

### Deletion Analysis of the Exemplary Polynucleotide Delivery-Enhancing Polypeptide

The efficacy of the full-length and truncated forms of polypeptide PN73 to enter cells was tested in vitro by a cell-uptake assay with primary mouse tail fibroblast (MTF) cells. The number of cells in culture that receive the FITC-labeled peptide was measured by flow cytometry. The percentage peptide cell-uptake was expressed relative to the total number of cells present in the culture. In addition, the Mean Fluorescence Intensity (MFI) was used to evaluate the quantity of FITC-labeled peptide found within cells. MFI directly correlates with the amount of FITC-labeled peptide within the cell: higher relative MFI value correlates with a greater amount of intracellular FITC-labeled peptides. Peptides were evaluated at 0.63 μM, 2.5 μM and 10 μM concentrations; PN768 was tested at 2 μM, 10 μM and 50 μM.

Full-length and truncated forms of the exemplary polynucleotide delivery-enhancing polypeptide PN73, were exposed to cells the day before transfection. FITC-tagged peptides were diluted in Opti-MEM® media (Invitrogen) for about 5 minutes at room temperature and then added to cells. Cells were transfected for 3 hours at and washed with PBS, treated with trypsin, and then analyzed by flow cytometry. Cell viability was determined as above. Cellular uptake was distinguished from the membrane insertion using trypan blue to quench any fluorescence on the cell membrane surface.

For the cell-uptake assay, the full-length FITC-labeled PN73 peptide (PN690) achieved nearly 100% cell uptake at all tested concentrations (10 μM results shown in Table 20 column entitled “% Peptide Cell-Uptake”). The remaining truncated forms of PN73, at 10 μM concentration except for PN768 which required 50 μM, achieved a percent cell uptake (values in parentheses) comparable to that of PN690 indicating that the N-terminal residues of PN73 are not required for the peptide's ability to enter cells. The five C-terminal residues of PN73, identified as PN768, are sufficient for peptide cell-uptake. The truncated forms of PN73 at 0.63 μM showed a decrease in cell uptake activity proportionate to the length of the peptide. In other words, the general observation of the peptides tested at a 0.63 μM concentration is that, as the PN73 peptide's length decreased, its cell uptake activity decreased thus indicating peptide cell-uptake activity is dose dependent.

Table 20 summarizes data for cell uptake and target gene knockdown (KD).

TABLE 20
Summary of Functional Domain Analysis of the PN73 Peptide Deletion Series

| C- | | | | | | |
|---|---|---|---|---|---|---|
| Term. | | % Peptide | Peptide | % siRNA | siRNA | |
| Label | Peptide | Cell-Uptake | FITC MFI | Cell-Uptake | Cy5 MFI | KD |
| None | PN73 | N/A | N/A | 98% (10 μM) | 13 (10 μM) | + |
| | PN360 | N/A | N/A | 0% | NT | NT |
| | PN361 | N/A | N/A | 55% (20 μM) | NT | NT |
| FITC (fluorescein-5- | PN690 | 100 (10 μM) | 125 (10 μM) | 58 (2.5 μM) | 50 (10 μM) | + |
| isothiocyanate) label | (PN73) | | | | | |
| (i.e., -GK[EPSILON]G- | PN661 | 100 (10 μM) | 128 (10 μM) | 49 (2.5 μM) | 59 (10 μM) | NT |
| amide) | PN685 | 100 (2.5 μM) | 151 (10 μM) | 24 (2.5 μM) | 61 (10 μM) | NT |
| | PN660 | 100 (10 μM) | 121 (10 μM) | 41 (2.5 μM) | 68 (10 μM) | + |
| | PN735 | 100 (10 μM) | 82 (10 μM) | 13 (2.5 μM) | 38 (10 μM) | − |
| | PN655 | 100 (10 μM) | 63 (10 μM) | 14 (10 μM) | 44 (10 μM) | NT |
| | PN654 | 95 (10 μM) | 10 (10 μM) | 27 (10 μM) | 14 (10 μM) | ± |
| | PN708 | 97 (10 μM) | 10 (10 μM) | 42 (10 μM) | 34 (10 μM) | + |
| | PN653 | 95 (10 μM) | 8 (10 μM) | 1.7 (10 μM) | 4 (10 μM) | − |
| | PN652 | 86 (10 μM) | 5 (10 μM) | 1.8 (10 μM) | 5 (10 μM) | NT |
| | PN651 | 90 (10 μM) | 5 (10 μM) | 0 | 3 (.65 μM) | NT |
| | PN768 | 91 (50 □M) | 9 (50 μM) | NT | NT | NT |

NT = not tested;
peptide concentrations (in parenthesis) given are those that achieved the given uptake, in percent, or MFI in relative values.

Table 20 shows that deleting part of the N-terminus of PN73 (see PN361) reduced siRNA cell-uptake activity by 50%; and removal of C-terminal residues (see PN360) reduced siRNA cell-uptake activity. These data show that the C-terminal domain of the exemplary polynucleotide deliver-enhancing polypeptide PN73 contributes to nucleotide cell-uptake activity of the peptide.

The effective knockdown of target gene expression by siRNA/polynucleotide delivery-enhancing polypeptide complexes of the invention was demonstrated. Specifically, the ability of siRNA/peptide complexes to modulate expression of the human tumor necrosis factor-α (hTNF-α) gene was assessed. The significance of targeting the hTNF-α gene is that it is implicated in mediating the occurrence or progression of rheumatoid arthritis (RA) when over-expressed in human and other mammalian subjects.

Human monocytes were used as a model system to determine the effect of siRNA/peptide complexes on hTNF-α gene expression. Qneg represents a random siRNA sequence and functioned as the negative control. The observed Qneg knockdown activity is normalized to 100% (100% gene expression levels) and the knockdown activity of each of the following siRNAs A19S21, 21/21 and LC20 was presented as a relative percentage of the negative control. A19S21, 21/21 and LC20 are siRNAs that target hTNF-α mRNA. The exemplary polynucleotide delivery-enhancing polypeptides PN643 (full-length PN73 minus a C-terminal label), PN690 (full-length PN73 with a C-terminal FITC-label) and the truncated forms of PN73 from the deletion series, PN660, PN735, PN654 and PN708 were complexed with the above listed siRNAs to determine their effect on each siRNA's ability to reduce hTNF-α gene expression levels in human monocytes. The knockdown activity for the full length and truncated forms of the exemplary polynucleotide delivery-enhancing polypeptide PN73 are summarized above in Table 20. A “+” in the “KD” column indicates that the peptide/siRNA complex had knockdown activity of 80% of the Qneg negative control siRNA (20% reduction in mRNA levels compared to the Qneg negative control). A “+/−” indicates that the peptide/siRNA complex had a knockdown activity of approximately 90% of the Qneg negative control siRNA (10% reduction in mRNA levels compared to the Qneg negative control). Finally, a “−” indicates that the peptide/siRNA complex had no significant knockdown activity compared to the Qneg negative control.

Healthy human blood was purchased from Golden West Biologicals (CA), the peripheral blood mononuclear cells (PBMC) were purified from the blood using Ficoll-Pague plus (Amersham) gradient. Human monocytes were then purified from the PBMCs fraction using magnetic microbeads from Miltenyi Biotech. Isolated human monocytes were resuspended in IMDM supplemented with 4 mM glutamine, 10% FBS, 1× non-essential amino acid and 1× pen-strep, and stored at 4 C until use.

In a 96 well flat bottom plate, human monocytes were seeded at 100 K/well/100 μl in OptiMEM medium (Invitrogen). Exemplary polynucleotide delivery-enhancing polypeptides were mixed with 20 nM siRNA at a molar ratio of 1 to 5 in OptiMEM medium at room temperature for 5 minutes. At the end of incubation, FBS was added to the mixture (final 3%), and 50 μl of the mixture was added to the cells. The cells were incubated at 37° C. for 3 hours. After incubation, the cells were transferred to V-bottom plate and pelleted at 1500 rpm for 5 min. The cells were resuspended in growth medium (IMDM with glutamine, non-essential amino acid, and pen-strep). After an overnight incubation, the monocytes were stimulated by application of LPS (Sigma) at 1 ng/ml for 3 hours to increase expression of TNF-α expression levels. After induction by LPS, cells were collected as above for mRNA quantification, and supernatant was saved for protein quantification if desired.

For mRNA measurement, branch DNA technology from Genospectra (CA) was used according to manufacturer's specification. To quantitate mRNA level in the cells, both house keeping gene (cypB) and target gene (TNF-α) mRNA were measured, and the reading for TNF-α was normalized with cypB to obtain relative luminescence unit.

In general, PN643 (full-length non-FITC-labeled PN73) and PN690 (full-length FITC-labeled PN73) had equivalent siRNA knockdown activities for all siRNAs tested as indicated by “+” in the “KD” column (results shown in Table 20). Additionally, PN660 had siRNA knockdown activities for all siRNAs tested that were comparable to PN643 and PN690 indicating that the removal of the 9 most N-terminal residues of the PN73 peptide did not affect siRNAs mediated knockdown activity of the targeted TNF-α mRNA. PN654 showed moderate knockdown activity for both the A19S21 and 21/21 siRNAs but not for the LC20 siRNA (knockdown activity is shown by “±” in knockdown activity column). However, the siRNAs complexed with either PN708 or PN735 resulted in no observable knockdown activity for any of the siRNAs.

### Example 15

### Polynucleotide Delivery-Enhancing Polypeptide PN708

As described above, the cell-uptake assay determines the number of cells that receive Cy5-conjugated siRNA when complexed with a peptide. siRNA cell-uptake was assessed by flow cytometry (refer to Example 2 for details). Uptake was expressed as a percentage calculated by dividing the number of cells containing Cy5-conjugated siRNA by the total number of transfected and untransfected cells in culture. Mean Fluorescence Intensity (MFI) was measured by flow cytometry and determined the amount of Cy5-conjugated siRNA found within cells. The MFI value directly correlates with the amount of Cy5-conjugated siRNA within the cell, thus, a higher MFI value indicates a greater number of Cy5-conjugated siRNA within the cells.

In this example, PN643 (full-length PN73 minus a C-terminal label), PN690 (full-length PN73 with a C-terminal FITC-label) and PN708 (15-mer derived by deletion of the 21 N-terminal residues of PN73) were tested at 5 μM, 10 μM, 20 μM and 40 μM. PN643 and PN690 were also tested at 2.5 μM and PN690 was additionally tested at 1.25 μM. PN643 and PN708 were also both tested at 80 μM.

As shown in Table 21, the non-FITC labeled PN73 (PN643) peptide achieved nearly 100% uptake of siRNA at 10 μM concentration. However, when the PN73 peptide was labeled with the FITC tag (PN690), its maximum cell-uptake activity was reduced to approximately 70%. PN708 showed a dose dependent increase in siRNA cell-uptake activity. PN708 achieved a maximum siRNA cell-uptake activity of 95% at 80 μM. For the full-length PN73 peptides, cell viability decreased as the concentration of peptide increased. In contrast, cells incubated with the PN708 peptide maintained over 90% cell viability in the presence of all tested concentrations. In this example, the truncated peptide PN708 about doubled the amount of Cy5-siRNA delivered into cells compared to the full-length PN73 (PN690) peptide.

TABLE 21
Summary of siRNA Delivery-Enhancing Characteristics of PN708

| | | | % siRNA | | |
|---|---|---|---|---|---|
| | Peptide | | Cell- | siRNA | % Cell |
| Treatment | Concentration | | Uptake | Cy5-MFI | Viability |
| Negative Control | 0 | | 0 | 0 | 98% |
| (no treatment) | | | | | |
| Cy5-LC20 siRNA + | 2.5 | μM | 61% | 8 | 95% |
| PN643 | 5 | μM | 96% | 13 | 95% |
| | 10 | μM | 97% | 17 | 94% |
| | 20 | μM | 84% | 10 | 93% |
| | 40 | μM | 44% | 7 | 78% |
| | 80 | μM | 12% | 14 | 26% |
| Cy5-LC20 siRNA + | 1.25 | μM | 30% | 7 | 95% |
| PN690 | 2.5 | μM | 47% | 17 | 97% |
| | 5 | μM | 71% | 56 | 94% |
| | 10 | μM | 64% | 67 | 92% |
| | 20 | μM | 55% | 90 | 91% |
| | 40 | μM | 45% | 218 | 71% |
| Cy5-LC20 siRNA + | 5 | μM | 35% | 9 | 96% |
| PN708 | 10 | μM | 55% | 23 | 96% |
| | 20 | μM | 83% | 85 | 97% |
| | 20 | μM | 93% | 212 | 94% |
| | 80 | μM | 96% | 378 | 91% |

Polypeptide PN708 was characterized by determining its affect on siRNA mediated target gene expression reduction. The C-terminal FITC-label of the PN708 peptide was removed prior to assessing its ability to enhance targeted gene expression reduction when complexed with a siRNA. In the absence of the FITC-label, the truncated exemplary polynucleotide delivery-enhancing polypeptide was named PN766 (refer to Table 19 in Example 12). The ability of siRNA/peptide complexes to modulate expression of the human tumor necrosis factor-α (hTNF-α) gene was assessed (protocol details can be found in Example 3). In this example, the random siRNA sequence, Qneg, served as a negative control and the siRNAs LC20 and LC 17 were used to target the hTNF-α mRNA in human monocytes. The molar ratios of siRNA to peptide tested were 1:5; 1:10; 1:25; 1:50; 1:75 and 1:100. Both LC20 and LC17 were used at 20 nM concentration.

The knockdown results were that both the LC20/PN766 and LC17/PN766 siRNA/peptide complexes at 1:5; 1:10; and 1:25 reduced hTNF-α mRNA levels to approximately 70%-80% of the Qneg siRNA negative control (i.e., 20%-30% reduction in mRNA levels compared to the Qneg negative control). The siRNA/peptide ratios of 1:50; 1:75 and 1:100 had no significant affect on hTNF-α mRNA levels compared to the Qneg control. No cytotoxicity effects were observed with human monocytes in the presence of the PN766 peptide.

### Example 16

### Peptide Mediated siRNA Cell-Uptake Activity

The siRNA cell-uptake assay and MFI measurements were performed as described previously in Examples 2 and 3. The data is summarized in Table 22. Each peptide was tested at 0.63 μM, 1.25 μM, 2.5 μM and 5 μM concentrations.

TABLE 22
Summary of PN73 Mutant Mediated siRNA Delivery Characteristics

| | | | % siRNA Cell- | siRNA | % Cell |
|---|---|---|---|---|---|
| Peptide | Concentration | | Uptake | Cy5 MFI | Viability |
| No Treatment | N/A | | 0% | 2 | 92% |
| PN73 | 0.63 | μM | 52% | 2 | 87% |
| | 1.25 | μM | 62% | 4 | 82% |
| | 2.5 | μM | 74% | 14 | 88% |
| | 5 | μM | 91% | 22 | 93% |
| PN644 | 0.63 | μM | 67% | 4 | 88% |
| | 1.25 | μM | 71% | 8 | 90% |
| | 2.5 | μM | 70% | 24 | 86% |
| | 5 | μM | 83% | 37 | 87% |
| PN645 | 0.63 | μM | 68% | 5 | 84% |
| | 1.25 | μM | 70% | 11 | 89% |
| | 2.5 | μM | 78% | 21 | 90% |
| | 5 | μM | 88% | 28 | 90% |
| PN646 | 0.63 | μM | 67% | 4 | 81% |
| | 1.25 | μM | 70% | 10 | 85% |
| | 2.5 | μM | 73% | 24 | 87% |
| | 5 | μM | 88% | 24 | 92% |
| PN647 | 0.63 | μM | 71% | 13 | 85% |
| | 1.25 | μM | 74% | 34 | 83% |
| | 2.5 | μM | 83% | 39 | 88% |
| | 5 | μM | 85% | 41 | 87% |
| PN729 | 0.63 | μM | 61% | 4 | 82% |
| | 1.25 | μM | 69% | 10 | 91% |
| | 2.5 | μM | 79% | 16 | 92% |
| | 5 | μM | 86% | 30 | 90% |

### Example 17

### Polynucleotide Delivery-Enhancing Polypeptides

Polynucleotide delivery-enhancing polypeptides shown in Table 23 were screened for their ability to deliver siRNA into mouse tail fibroblast (MTF) cells.

TABLE 23
Delivery-Enhancing Polypeptides
Screened for siRNA Cell-Uptake Activity

| Peptide | Amino Acid Sequence | Name |
|---|---|---|
| PN680 | (SEQ ID NO: 87) | Androctonin |
| | RSVCRQIKICRRRGGCYYKCTNRPY- | |
| | amide | |
| PN665 | (SEQ ID NO: 88) | Paradaxin |
| | GFFALIPKIISSPLFKTLLSAVGSAL | |
| | SSSGDQE-amide | |
| PN734 | (SEQ ID NO: 89) | m-Calpain + |
| | GTAMRILGGVIPRKKRRQRRRPPQ- | TAT |
| | amide | |
| PN681 | (SEQ ID NO: 90) | MARCKS |
| | KKKKKRFSFKKSFKLSGFSFKKNKK- | |
| | amide | |
| PN694 | (SEQ ID NO: 91) | Penetratin |
| | RQIKIWFQNRRMKWKK-amide | |
| PN714 | (SEQ ID NO: 92) | PenArg |
| | RQIRIWFQNRRMRWRR-amide | |
| PN760 | (SEQ ID NO: 93) | TAT + |
| | RKKRRQRRRPPVAYISRGGVSTYYSD | Peptide |
| | TVKGRFTRQKYNKRA-amide | P3a |
| PN759 | (SEQ ID NO: 94) | Bindin + |
| | LGLLLRHLRHHSNLLANIPRKKRRQR | TAT |
| | RRPP-amide | |
| PN682 | (SEQ ID NO: 95) | Pep-1 |
| | KETWWETWWTEWSQPKKKRKV- | |
| | amide | |

The siRNA cell-uptake activity for the polynucleotide delivery-enhancing polypeptides listed in Table 23 complexed with siRNA. Table 24 summarizes the siRNA cell-uptake data, mean fluorescence intensity (MFI) measurements and cell viability data for each of the polypeptides. Polypeptides that achieved a percent siRNA cell-uptake of 75% or greater are highlighted in grey in the “Treatment” column. The specific percent siRNA cell-uptake for each these highlighted siRNA/peptide complexes is also highlighted in grey in the “% siRNA Cell-Uptake” column.

LC20 is an oligo used for the siRNA targeting of the human tumor necrosis factor-alpha (hTNF-α) mRNA and is represented by the ribonucleotide sequence:

(SEQ ID NO: 96)
UAGGGUCGGAACCCAAGCUUA

siRNA uptake by cells was assessed by flow cytometry (refer to Example 2 for details). Uptake was expressed as a percentage calculated by dividing the number of cells containing Cy5-conjugated siRNA by the total number of transfected and untransfected cells in culture. Mean Fluorescence Intensity (MFI) was measured by flow cytometry and determined the amount of Cy5-conjugated siRNA found within cells. The MFI value directly correlates with the amount of Cy5-conjugated siRNA within the cell, thus, a higher MFI value indicates a greater number of Cy5-conjugated siRNA within the cells.

The data show that PN680, PN681, PN709, PN760, PN759, and PN682, when complexed with siRNA, deliver siRNA into cells. The results for the screening of polypeptides shown in Table 23 are shown in Table 24.

TABLE 24
Data of Polypeptide Mediated siRNA
Delivery Screen (NT = not tested)

| Treatment | | | % siRNA | Cy5- | |
|---|---|---|---|---|---|
| siRNA/peptide | Peptide | | Cell- | siRNA | % Cell |
| Complex | Concentration | | Uptake | MFI | Viability |
| No treatment | N/A | | 0.0% | 0.0 | 97.6% |
| Cy5-LC20 + PN643 | 5 | μM | 95.4% | 7.2 | 98.8% |
| (positive control) | | | | | |
| Cy5-LC20 + PN680 | 0.63 | μM | 0.2% | N/T | 98.2% |
| | 2.5 | μM | 1.8% | 1.4 | 98.3% |
| | 10 | μM | 82.6% | 4.5 | 99.2% |
| | 40 | μM | 79.1% | 5.2 | 95.7% |
| Cy5-LC20 + PN665 | 0.63 | μM | 0.0% | N/T | 97.7% |
| | 2.5 | μM | 0.6% | N/T | 95.1% |
| | 10 | μM | N/T | N/T | N/T |
| | 40 | μM | N/T | N/T | N/T |
| Cy5-LC20 + PN734 | 0.63 | μM | 0.1% | N/T | 98.2% |
| | 2.5 | μM | 0.2% | N/T | 98.7% |
| | 10 | μM | 1.2% | 1.3 | 98.4% |
| | 40 | μM | 4.5% | 1.6 | 97.0% |
| Cy5-LC20 + PN681 | 0.63 | μM | 0.2% | 1.8 | 97.1% |
| | 2.5 | μM | 69.9% | 4.6 | 98.9% |
| | 10 | μM | 97.3% | 15.3 | 98.2% |
| | 40 | μM | 91.2% | 13.7 | 92.6% |
| Cy5-LC20 + PN694 | 0.63 | μM | 0.2% | 1.4 | 97.1% |
| | 2.5 | μM | 0.2% | 1.8 | 97.9% |
| | 10 | μM | 48.0% | 4.2 | 97.8% |
| | 40 | μM | 54.0% | 3.9 | 83.6% |
| Cy5-LC20 + PN714 | 0.63 | μM | 0.4% | 1.2 | 95.1% |
| | 2.5 | μM | 0.5% | 2.3 | 96.4% |
| | 10 | μM | 19.1% | 2.5 | 97.6% |
| | 40 | μM | 43.0% | 4.9 | 94.7% |
| Cy5-LC20 + PN709 | 0.63 | μM | 0.1% | 1.0 | 94.0% |
| | 2.5 | μM | 0.2% | 1.0 | 96.6% |
| | 10 | μM | 18.6% | 1.9 | 97.1% |
| | 40 | μM | 76.6% | 5.8 | 97.1% |
| Cy5-LC20 + PN760 | 0.63 | μM | 60% | 2.9 | 84.7% |
| | 2.5 | μM | 85.5% | 78.5 | 90.8% |
| | 10 | μM | 90.6% | 96.9 | 91.9% |
| | 40 | μM | 82.8% | 77.4 | 83.2% |
| Cy5-LC20 + PN759 | 0.63 | μM | 43% | 2.1 | 81.7% |
| | 2.5 | μM | 72.9% | 7.3 | 85.2% |
| | 10 | μM | 83.6% | 40.9 | 86.7% |
| | 40 | μM | 25% | 10.5 | 26.6% |
| Cy5-LC20 + PN682 | 0.63 | μM | 52.1% | 2.4 | 86.2% |
| | 2.5 | μM | 50.6% | 2.2 | 91.3% |
| | 10 | μM | 56.9% | 2.3 | 90.6% |
| | 40 | μM | 92% | 9.0 | 97.1% |

As shown in the column entitled “% siRNA Cell-Uptake” of Table 24, the “no treatment” negative control showed no siRNA cell-uptake while the positive control peptide achieved a percent siRNA cell-uptake activity of 95%. The Cy5 conjugated LC20 siRNA complexed with the polynucleotide delivery-enhancing polypeptides PN680; PN681; PN709; PN760; PN759 or PN682 achieved a percent siRNA cell-uptake activity that exceeded 75% or greater. The polypeptides PN694 and PN714 exhibited a moderate siRNA cell-uptake activity of 54% and 43%, respectively. The polypeptides PN665 and PN734 demonstrated no significant siRNA cell-uptake activity (less than 5%).

The polypeptides were further characterized for their ability to transfect siRNAs into cells by analyzing Mean Fluorescence Intensity (MFI). While the cell-uptake assay determined the percentage of cells that contain the Cy5-conjugated siRNA, the MFI measurement determined the relative mean quantity of Cy5-conjugated siRNA that entered the cells. As shown in the column entitled “siRNA Cy5 MFI” of Table 24, delivery of the Cy5-conjugated siRNA by the positive control peptide PN643 achieved a MFI of approximately seven units. As expected, the “no treatment” negative control has no measurable MFI. The polynucleotide delivery-enhancing polypeptide PN665 was not tested by MFI. PN743, PN694 and PN714 had MFI measurements significantly lower than that of the positive control. The polynucleotide delivery-enhancing polypeptides PN680, PN709 and PN682 exhibited MFI measurements comparable to that of the PN643 positive control while PN681 had an MFI double that of the positive control. PN760 and PN759 had MFI measurements that were approximately 13-fold and 6-fold greater, respectively, than that of the positive control.

The following protocol was used to test the polynucleotide delivery-enhancing polypeptides listed in Table 23. Approximately 80,000 mouse tail fibroblast (MTF) cells were plated per well in 24-well plates the day before transfection in complete media. Each delivery peptide, except the positive control, was tested at 0.63 μM, 2.5 μM, 10 μM and 40 μM concentrations in the presence of 0.5 μM Cy5-conjugated siRNA. For siRNA/peptide complexes, the Cy5-conjugated siRNA and peptide were diluted separately in Opti-MEM® media (Invitrogen) at two-fold the final concentration. Equal volumes of siRNA and peptide were mixed and allowed to complex five minutes at room temperature. The siRNA/peptide complexes were added to cells previously washed with phosphate buffered saline (PBS). Cells were transfected for three hours at 37° C., 5% CO₂. Cells were washed with PBS, treated with trypsin, and then analyzed by flow cytometry. siRNA cell-uptake was measured by the intensity of intracellular Cy5 fluorescence. Cell viability was determined using propidium iodide uptake or AnnexinV-PE (BD Biosciences) staining. In order to differentiate the cellular uptake from the membrane insertion of labeled siRNA (or fluorescein-labeled peptide), trypan blue was used to quench any fluorescence on the cell membrane surface. Trypan blue (Sigma) was added to cells to a final concentration of 0.04% and re-run on the flow cytometer to assess whether there was any change in fluorescence intensity which would indicate fluorescence localized to the cell membrane.

### Example 18

### Knockdown Activity of siRNAs With Polypeptides

The ability of siRNA/peptide complexes to modulate expression of the human tumor necrosis factor-α (hTNF-α) gene was assessed.

Human monocytes were used as a model system to determine the effect of siRNA/peptide complexes on hTNF-α gene expression. Qneg represents a random siRNA sequence and functioned as the negative control. The observed Qneg knockdown activity was normalized to 100% (100% gene expression levels) and the knockdown activity for each of the following siRNAs A19S21 MD8, 21/21 MD8 and LC20 was presented as a relative percentage of the negative control. A19S21 MD8, 21/21 MD8 and LC20 are siRNAs that target hTNF-α mRNA.

The polypeptide PN602 is an acetylated form of the positive control used in prior Examples and was used in this example as a positive control for both the effective delivery of siRNA into human monocytes and the permissive knockdown activity of hTNF-α mRNA levels mediated by siRNA.

The data show that the polynucleotide delivery-enhancing polypeptide PN680 delivers siRNAs into cells and permits effective siRNA mediated gene silencing. The knockdown activity of PN602, PN680, and PN681 is shown in Table 25. A “+” symbol indicates that the peptide/siRNA complex had knockdown activity of 80% of the Qneg negative control siRNA (20% reduction in mRNA levels compared to the Qneg negative control). A “+/−” indicates that the peptide/siRNA complex had a knockdown activity of approximately 90% of the Qneg negative control siRNA (10% reduction in mRNA levels compared to the Qneg negative control). Finally, a “−” indicates that the peptide/siRNA complex had no significant knockdown activity compared to the Qneg negative control.

TABLE 25
siRNA Knockdown Activity for siRNAs Complexed With Polypeptides

| | | siRNA | | |
|---|---|---|---|---|
| | siRNA:Peptide | A19S21 | 21/21 | |
| Peptide ID # | Ratio | MD8 | MD8 | LC20 |
| PN602 | 1:5 | +/− | +/− | +/− |
| (Positive control) | 1:10 | +/− | +/− | +/− |
| PN680 | 1:5 | + | + | + |
| | 1:10 | +/− | +/− | + |
| PN681 | 1:5 | +/− | − | − |
| | 1:10 | +/− | − | − |

The results shown in Table 25 indicate that all three siRNAs complexed with the positive control PN602 polynucleotide delivery-enhancing polypeptide at ratios of 1:5 and 1:10 moderately reduced hTNF-α gene expression levels compared to the Qneg negative control complexed with the same polypeptide. However, the same siRNAs complexed with the polynucleotide delivery-enhancing polypeptide PN681 at 1:5 and 1:10 showed little to no knockdown activity relative to the Qneg negative control siRNA/PN681 complex. In contrast, the polynucleotide delivery-enhancing polypeptide PN6.80 complexed with any of the hTNF-α specific siRNAs at a 1:5 ratio exhibited significant knockdown activity of the hTNF-α mRNA relative to the Qneg/PN680 control complex. Furthermore, the LC20/PN680 complex at a 1:10 ratio also demonstrated significant knockdown activity compared to the Qneg/PN680 control complex.

Healthy human blood was purchased from Golden West Biologicals (CA), the peripheral blood mononuclear cells (PBMC) were purified from the blood using Ficoll-Pague plus (Amersham) gradient. Human monocytes were then purified from the PBMCs fraction using magnetic microbeads from Miltenyi Biotech. Isolated human monocytes were resuspended in IMDM supplemented with 4 mM glutamine, 10% FBS, 1× non-essential amino acid and 1× pen-strep, and stored at 4 C until use.

In a 96 well flat bottom plate, human monocytes were seeded at 100 K/well/100 μl in OptiMEM medium (Invitrogen). The polynucleotide delivery-enhancing polypeptides were mixed with 20 nM siRNA at a molar ratio of 1:5 or 1:10 in OptiMEM medium at room temperature for five minutes. At the end of incubation, FBS was added to the mixture (final 3%), and 50 μl of the mixture was added to the cells. The cells were incubated at 37° C. for 3 hours. After incubation, the cells were transferred to V-bottom plate and pelleted at 1500 rpm for five minutes. The cells were resuspended in growth medium (IMDM with glutamine, non-essential amino acid, and pen-strep). After an overnight incubation, the monocytes were stimulated by application of LPS (Sigma) at 1 ng/ml for three hours to increase expression of TNF-α expression levels. After induction by LPS, cells were collected as above for mRNA quantification, and supernatant was saved for protein quantification if desired.

For mRNA measurement, branch DNA technology from Genospectra (CA) was used according to manufacturer's specification. To quantitate mRNA level in the cells, both house keeping gene (cypB) and target gene (TNF-α) mRNA were measured, and the reading for TNF-α was normalized with cypB to obtain relative luminescence unit.

What is claimed is:

1. A compound comprising condensed particles having diameters less than 1000 nm, wherein the particles comprise one or more double stranded ribonucleic acids (dsRNAs) and one or more peptides.

2. The compound of claim 1, wherein the diameters are from 0.5-400 nm.

3. The compound of claim 1, wherein the diameters are from 10-300 nm.

4. The compound of claim 1, wherein the diameters are from 40-100 nm.

5. The compound of claim 1, wherein the peptides are from 5-99% of the mass of the particles.

6. The compound of claim 1, wherein the peptides are from 5-50% of the mass of the particles.

7. The compound of claim 1, wherein the peptides are from 50-99% of the mass of the particles.

8. The compound of claim 1, wherein the particles are uniform.

9. The compound of claim 1, wherein the particles are spherical of uniform diameter.

10. The compound of claim 1, wherein the particles have a zeta potential magnitude of at least 20 mV.

11. The compound of claim 1, wherein the particles have a zeta potential magnitude of at least 30 mV.

12. The compound of claim 1, wherein the diameter is from 10-300 nm and the dsRNA is a siNA targeted to influenza virus.

13. The compound of claim 1, wherein the diameter is from 10-300 nm and the dsRNA is a siNA targeted to human TNF-α.

14. The compound of claim 1, wherein the compound releases a dsRNA intracellularly to inhibit gene expression in a cell.

15. The compound of claim 1, wherein the particles are crosslinked.

16. The compound of claim 1, wherein the particles are prepared by spray drying.

17. A compound made by the method comprising:
providing one or more double stranded ribonucleic acids (dsRNAs) and one or more peptides; and
condensing the dsRNAs with the peptides in an aqueous solution thereby forming particles having diameters less than 1000 nm.

18. The compound of claim 17, wherein the charge ratio N/P for each peptide is from 0.2 to 50.

19. The compound of claim 17, wherein the charge ratio N/P for each peptide is from 0.5 to 20.

20. The compound of claim 17, wherein the charge ratio N/P for each peptide is from 0.5 to 7.

21. The compound of claim 17, wherein the charge ratio N/P for each peptide is from 0.5 to 2.5.

22. The compound of claim 17, wherein the diameter is from 10-300 nm and the dsRNA is a siNA targeted to influenza.

23. The compound of claim 17, wherein the particles have a zeta potential magnitude of at least 20 mV.

24. The compound of claim 17, wherein the aqueous solution has a pH of less than or about equal to 11.

25. The compound of claim 17, wherein the aqueous solution has a pH of less than or about equal to 9.

26. The compound of claim 17, wherein the aqueous solution has a pH of less than or about equal to 8.

27. The compound of claim 17, wherein the aqueous solution is vortexed.

28. The compound of claim 17, wherein the condensing is performed by adding the peptides to an aqueous solution of the dsRNA molecules.

29. The compound of claim 17, wherein the aqueous solution contains a salt.

30. The compound of claim 29, wherein the aqueous solution has a sodium chloride concentration of less than or equal to about 1 M.

31. The compound of claim 29, wherein the aqueous solution has a sodium chloride concentration of less than or equal to about 0.5 M.

32. The compound of claim 29, wherein the aqueous solution has a sodium chloride concentration of less than or equal to about 0.25 M.

33. The compound of claim 1, wherein the ribonucleic acid agent is a siNA.

34. The compound of claim 1, wherein the ribonucleic acid agent is a siNA targeted to influenza or TNF-α.

35. The compound of claim 1, wherein the ribonucleic acid agent is selected from G1498, G8286, G8282, G6129, G6124, G3817, G3807, G3789, and combinations thereof.

36. The compound of claim 1, wherein the ribonucleic acid agent is G1498.

37. The compound of claim 1, wherein the compound decreases viral growth of an influenza virus by two-fold compared to the dsRNA alone.

38. The compound of claim 1, wherein each peptide has a mass of less than 120 kDa.

39. The compound of claim 1, wherein each peptide has a mass of less than 60 kDa.

40. The compound of claim 1, wherein each peptide has a mass of less than 30 kDa.

41. The compound of claim 1, wherein the number of positive charges for a peptide is from 1 to 100.

42. The compound of claim 1, wherein the number of positive charges for a peptide is from 5 to 30.

43. The compound of claim 1, wherein the number of positive charges for a peptide is from 9 to 15.

44. The compound of claim 1, wherein at least one of the peptides contains a protein transduction domain.

45. The compound of claim 1, wherein at least one of the peptides is a mucosal permeability modulator.

46. The compound of claim 1, wherein at least one of the peptides is pegylated.

47. The compound of claim 1, wherein at least one of the peptides is selected from PN183, PN826, PN861, PN924, PN939, and variants thereof.

48. The compound of claim 1, wherein at least one of the peptides is PN183.

49. A method of making a compound comprising the steps of:
providing one or more double stranded ribonucleic acids (dsRNAs) and one or more peptides; and
condensing the dsRNAs with the peptides in an aqueous solution thereby forming particles having diameters less than 1000 nm.

50. The method of claim 49, wherein the particles are crosslinked.

51. A method of making a compound comprising the steps of:
(a) providing one or more double stranded ribonucleic acids (dsRNAs) and one or more peptides;
(b) dissolving the dsRNAs in an aqueous solution; and
(c) adding the peptides to the aqueous solution thereby condensing particles having diameters less than 1000 nm.

52. A method of making a compound comprising the steps of:
(a) providing one or more double stranded ribonucleic acids (dsRNAs) and one or more peptides;
(b) dissolving the peptides in an aqueous solution; and
(c) adding the dsRNAs to the aqueous solution thereby condensing particles having diameters less than 1000 nm.

53. A pharmaceutical composition comprising a compound of claim 1 and a carrier vehicle.

54. A method of treating influenza comprising administering to a subject in need a therapeutically effective amount of a pharmaceutical composition containing a compound of claim 1 wherein the ribonucleic acid agent is a siNA targeted to influenza.

55. The method of claim 54, wherein the siNA is 01498.

56. A use of the compound of claim 1 as a medicament for treating the signs and symptoms of a disease or condition in a human including influenza and rheumatoid arthritis.

57. A use of the compound of claim 1 in the manufacture of a medicament for treating the signs and symptoms of a disease or condition in a human including influenza and rheumatoid arthritis.

58. A use of a compound comprising condensed particles having diameters less than 1000 nm, wherein the particles comprise one or more double stranded ribonucleic acids (dsRNAs) and one or more peptides in the manufacture of a medicament for treating the signs and symptoms of a disease or condition in a human including influenza and rheumatoid arthritis.

59. The use of claim 58, wherein the diameters are from 10-300 nm and the dsRNA is a siNA targeted to influenza virus.

60. The use of claim 58, wherein the compound releases a dsRNA intracellularly to inhibit gene expression in a cell.
